(19) United States
(12) Patent Application Publication
(10) Pub. No.: US 2024/0075271 A1
(43) Pub. Date: Mar. 7, 2024

(54) Wireless Communication and Power Conservation for Implantable Monitors

(71) Applicant: Veris Health, Inc., New York, NY (US)

(72) Inventors: James D. Mitchell, New York, NY (US); Andrew Thoreson, New York, NY (US); Theodore C. Johnson, New York, NY (US); Jayme Ormiston Coates, New York, NY (US)

(21) Appl. No.: 18/268,930

(22) PCT Filed: Dec. 20, 2021
(86) PCT No.: PCT/US21/73041

§ 371 Date: Jun. 21, 2023

Related U.S. Application Data
(60) Provisional application No. 63/199,360, filed on Dec. 21, 2020.

(51) Int. Cl. A61M 39/02; G16H 40/67

(52) U.S. Cl. CPC A61M 39/0247; G16H 40/67; A61M 2039/0258; A61M 2039/0267; A61M 2039/0288; A61M 2205/3507

(57) ABSTRACT

Implantable devices and associated devices, systems, and methods are disclosed herein. The devices and systems of the present technology may be equipped with electronic components that provide a platform for remote patient and/or device monitoring. Operation of an implantable devices and/or one or more components thereof can be modulated over time depending on certain conditions. This modulation can include limiting wireless data communication with external devices to certain time intervals, varying parameters of data collection by sensing elements, and/or controlling power supplied to electronic components, for example. In some examples, an implantable device may operate in a low-power standby state in which data is obtained via sensing elements and stored in local data storage, but the data is not wirelessly transmitted to an external device until a modulation signal is received by the implantable device, causing the implantable device to change to a more active power state.

### CROSS-REFERENCE TO RELATED APPLICATION(S)

This application claims the benefit of priority of U.S. Provisional Patent Application No. 63/199,360, filed Dec. 21, 2020, which is hereby incorporated by reference in its entirety.

### TECHNICAL FIELD

The present technology relates to implantable medical devices and associated systems and methods of use.

### BACKGROUND

Vascular access devices (e.g., vascular access ports) are minimally invasive, surgically implanted devices that provide relatively quick and easy access to a patient's central venous system for the purpose of administering intravenous medications, such as chemotherapeutic agents. Conventional vascular access devices are commonly used for patients requiring frequent, repeated intravenous administration of therapeutic agents or fluid, repeated blood draws, and/or for patients with difficult vascular access.

Vascular access devices include a myriad of typically catheter related devices meant to establish direct communication with the blood stream. Such devices include, but are not limited to, vascular access ports (VAPs), peripheral inserted central catheters (PICCs), midlines, peripheral IVs, amongst others. For purposes of simplicity, the present disclosure refers in several places throughout to vascular access ports (VAPs), or “ports” in common nomenclature.

Vascular access devices typically include a reservoir attached to a catheter. The entire unit is placed completely within a patient's body using a minimally invasive surgical procedure. In most cases the reservoir is placed in a small pocket created in the upper chest wall just inferior to the clavicle, and the catheter is inserted into the internal jugular vein or the subclavian vein with the tip resting in the superior vena cava or the right atrium. However, vascular access devices can be placed in other parts of the body and/or with the catheter positioned in alternative sites as well. In conventional devices, the reservoir is typically bulky such that the overlying skin protrudes, allowing a clinician to use palpation to localize the device for access when it is to be used for a medication infusion or aspiration of blood for testing. A self-sealing cover (e.g., a thick silicone membrane) is disposed over and seals the reservoir, allowing for repeated access using a non-coring (e.g., Huber type) needle that is inserted through the skin and into the port. This access procedure establishes a system in which there is fluid communication between the needle, the vascular access device, the catheter, and the vascular space, thereby enabling infusion of medication or aspiration of blood via a transcutaneous needle.

Conventional vascular access devices are bulky by design to allow a clinician to localize the device by palpation. To be accurately accessed by a clinician, the vascular access device needs to be either visualized or palpated under the skin. Additionally, conventional vascular access ports have no electronic components and no internal power source. Accordingly, there is a need for improved vascular access devices.

### SUMMARY

The present technology is directed to vascular access devices and mechanisms wireless communication and power conservation for such devices. The subject technology is illustrated, for example, according to various aspects described below, including with reference to FIGS. 1-14D. Various examples of aspects of the subject technology are described as numbered clauses (1, 2, 3, etc.) for convenience. These are provided as examples and do not limit the subject technology.
1. An implantable vascular access device comprising:
   a fluid reservoir;
   a cover disposed over the reservoir;
   an outlet port configured to mate with a catheter, the outlet port fluidically coupled to the fluid reservoir;
   one or more sensors configured to capture physiological data while the device is implanted within a patient;
   a first wireless transceiver configured to transmit the physiological data to one or more external devices via a first communication link; and
   a second wireless transceiver configured to communicate with one or more external devices via a second communication link;
   wherein the device is configured to transition between a low-power first state and a higher-power second state.
2. The device of Clause 1, wherein the first communication link comprises at least one of: a Bluetooth or a WiFi link.
3. The device of any one of the preceding Clauses, wherein the second communication network comprises a near-field communication (NFC) link.
4. The device of any one of the preceding Clauses, wherein the first wireless transceiver comprises a Bluetooth transceiver.
5. The device of any one of the preceding Clauses, wherein the first wireless transceiver is inactive while the device is in the first state, and wherein the first wireless transceiver is active while the device is in the second state.
6. The device of any one of the preceding Clauses, wherein the first wireless transceiver transmits data only while the device is in the second state.
7. The device of any one of the preceding Clauses, wherein the first state is a lower-power standby state, and wherein the second state is a higher-power operational state.
8. The device of any one of the preceding Clauses, wherein, in the standby state, the device does not transmit data via the first wireless transceiver, and wherein in the operational state, the device transmits data via the first wireless transceiver.
9. The device of any one of the preceding Clauses, wherein the device is configured to transition between the first state and the second state after a trigger event.
10. The device of any one of the preceding Clauses, wherein the trigger event comprises a measurement of a physiological parameter that falls above or below a predetermined threshold.
11. The device of any one of the preceding Clauses, wherein the trigger event comprises a measurement of a physiological parameter that falls outside of a predetermined range.
12. The device of any one of the preceding Clauses, wherein the trigger event comprises elapsing of a predetermined time.
13. The device of any one of the preceding Clauses, wherein the trigger event comprises receiving a modulation signal from an external device.
14. The device of any one of the preceding Clauses, wherein the modulation signal comprises a wireless signal received via an NFC coil.
15. The device of any one of the preceding Clauses, wherein the device is configured to transition from the second state back to the first state after a second trigger event.
16. The device of any one of the preceding Clauses, wherein the second trigger event comprises a measurement of a physiological parameter that falls within a predetermined range.
17. The device of any one of the preceding Clauses, wherein the second trigger event comprises a measurement of a physiological parameter that falls above or below a predetermined threshold.
18. The device of any one of the preceding Clauses, wherein the second trigger event comprises a measurement of a physiological parameter that changes at a rate that is higher or lower than a predetermined threshold.
19. The device of any one of the preceding Clauses, wherein the second trigger event comprises elapsing of a predetermined time.
20. The device of any one of the preceding Clauses, wherein the second trigger event comprises completion of data transmission to an external device.
21. The device of any one of the preceding Clauses, wherein, in the first state, at least one sensing element has a first sampling frequency and, in the second state, the at least one sensing element has a second sampling frequency greater than the first.
22. The device of any one of the preceding Clauses, wherein the sensors comprise one or more of: EKG sensors, a temperature sensor, an accelerometer, a gyroscope, a magnetometer, a pulse oximeter, a pressure sensor, a light sensor, a pH sensor, a blood-gas sensor, blood count sensor, or a blood-chemistry sensor.
23. The device of any one of the preceding Clauses, wherein the physiological data comprises one or more of: an EKG reading, a pulse rate, a blood pressure, a temperature, detected-motion data, a blood oxygenation, pH data, or blood-constituent data.
24. The device of any one of the preceding Clauses, further comprising a catheter fluidically coupled to the reservoir.
25. The device of any one of the preceding Clauses, wherein the first wireless link comprises one or more of: near-field communication (NFC), infrared wireless, Bluetooth, ZigBee, Wi-Fi, inductive coupling, or capacitive coupling.
26. The device of any one of the preceding Clauses, wherein the second wireless link comprises one or more of: near-field communication (NFC), infrared wireless, Bluetooth, ZigBee, Wi-Fi, inductive coupling, or capacitive coupling.
27. A method comprising:
   receiving, at a first wireless transceiver of an implantable monitor, a first signal over a first communication link;
   after receiving the first signal, transitioning the implantable monitor from a low-power first state to a higher-power second state;
   sensing at least one physiological parameter while implanted within the patient; and
   while in the second state, transmitting data associated with the at least one physiological parameter to one or more external devices over a second communication link via a second wireless transceiver different from the first.
28. The method of Clause 27, wherein the first communication link comprises at least one of: a Bluetooth or a WiFi link.
29. The method of any one of the preceding Clauses, wherein the second communication network comprises a near-field communication (NFC) link.
30. The method of any one of the preceding Clauses, wherein the first wireless transceiver comprises a Bluetooth transceiver.
31. The method of any one of the preceding Clauses, wherein the first wireless transceiver is inactive while the device is in the first state, and wherein the first wireless transceiver is active while the device is in the second state.
32. The method of any one of the preceding Clauses, wherein the first wireless transceiver transmits data only while the device is in the second state.
33. The method of any one of the preceding Clauses, wherein the first state is a lower-power standby state, and wherein the second state is a higher-power operational state.
34. The method of any one of the preceding Clauses, wherein, in the standby state, the device does not transmit data via the first wireless transceiver, and wherein in the operational state, the device transmits data via the first wireless transceiver.
35. The method of any one of the preceding Clauses, wherein the device transitions between the first state and the second state after a trigger event.
36. The method of any one of the preceding Clauses, wherein the trigger event comprises a measurement of a physiological parameter that falls above or below a predetermined threshold.
37. The method of any one of the preceding Clauses, wherein the trigger event comprises a measurement of a physiological parameter that indicates a rate of change that is above or below a predetermined threshold.
38. The method of any one of the preceding Clauses, wherein the trigger event comprises a measurement of a physiological parameter that falls outside of a predetermined range.
39. The method of any one of the preceding Clauses, wherein the trigger event comprises elapsing of a predetermined time.
40. The method of any one of the preceding Clauses, wherein the trigger event comprises receiving a modulation signal from an external device.
41. The method of any one of the preceding Clauses, wherein the modulation signal comprises a wireless signal received via an NFC coil.
42. The method of any one of the preceding Clauses, further comprising transitioning from the second state back to the first state after a second trigger event.
43. The method of any one of the preceding Clauses, wherein the trigger event comprises a measurement of a physiological parameter that falls within a predetermined range.
44. The method of any one of the preceding Clauses, wherein the trigger event comprises a measurement of a physiological parameter that falls above or below a predetermined threshold.
45. The method of any one of the preceding Clauses, wherein the trigger event comprises elapsing of a predetermined time.
46. The method of any one of the preceding Clauses, wherein the trigger event comprises completion of data transmission to an external device.
47. The method of any one of the preceding Clauses, wherein, in the first state, at least one sensing element has a first sampling frequency and, in the second state, the at least one sensing element has a second sampling frequency greater than the first.
48. The method of any one of the preceding Clauses, wherein the sensors comprise one or more of: EKG sensors, a temperature sensor, an accelerometer, a gyroscope, a magnetometer, a pulse oximeter, a pressure sensor, a light sensor, a pH sensor, a blood-gas sensor, or a blood-chemistry sensor.
49. The method of any one of the preceding Clauses, wherein the physiological data comprises one or more of: an EKG reading, a pulse rate, a blood pressure, a temperature, detected-motion data, a blood oxygenation, pH data, or blood-constituent data.
50. The method of any one of the preceding Clauses, wherein the first wireless link comprises one or more of: near-field communication (NFC), infrared wireless, Bluetooth, ZigBee, Wi-Fi, inductive coupling, or capacitive coupling.
51. The method of any one of the preceding Clauses, wherein the second wireless link comprises one or more of: near-field communication (NFC), infrared wireless, Bluetooth, ZigBee, Wi-Fi, inductive coupling, or capacitive coupling.

### BRIEF DESCRIPTION OF THE DRAWINGS

FIG. 1 is a schematic representation of a monitoring system in accordance with the present technology.

FIG. 2 shows an example of a vascular access device configured for use with the system of FIG. 1 in accordance with the present technology.

FIG. 3 shows the vascular access device of FIG. 2 implanted within a patient's body.

FIG. 4 is a schematic block diagram of an environment for communication between an implantable device, an interrogation device, and one or more remote computing devices in accordance with the present technology.

FIG. 5 is a schematic block diagram of an environment for communication between an implantable device and an interrogation device in accordance with the present technology.

FIG. 6A shows a schematic top view of an antenna in accordance with the present technology.

FIG. 6B shows a schematic top view of a circuit board in accordance with the present technology.

FIG. 6C shows a schematic side view of an electronics assembly in accordance with the present technology.

FIG. 7A shows a schematic top view of an antenna in accordance with the present technology.

FIG. 7B shows a schematic top view of a circuit board in accordance with the present technology.

FIG. 7C shows a schematic side view of an electronics assembly in accordance with the present technology.

FIG. 8 illustrates various example operational power states for an implantable device in accordance with the present technology.

FIG. 9 is a schematic block diagram of an environment for communication between an implantable device and an interrogation device in accordance with the present technology.

FIG. 10 is a flow diagram of an example process of obtaining and transmitting data in accordance with the present technology.

FIG. 11 is a schematic diagram of an implantable device in communication with an interrogation device in accordance with the present technology.

FIG. 12 is a flow diagram illustrating an example method of operation of an implantable device in accordance with the present technology.

FIGS. 13A-13C illustrate example graphical user interfaces of a software application for initiating a health check via an implantable device.

FIGS. 14A-14D illustrate example graphical user interfaces of a software application for querying a patient for information relevant to treatment.

### DETAILED DESCRIPTION

The devices and systems of the present technology may be equipped with electronic components that provide a platform for remote patient and/or device monitoring. Although vascular access devices are described by way of example throughout this disclosure, aspects of the present technology can be embodied in any suitable implantable medical device. In various embodiments, the vascular access devices disclosed herein may include a sensing element configured to obtain data characterizing a patient's health, performance of the device, treatment status, and/or other parameters for enhancing patient care. For example, a sensing element of an implantable device can be configured to obtain patient physiological data while the vascular access device is implanted within the patient. Devices of the present technology can be configured to determine (e.g., calculate or otherwise generate or obtain) one or more parameters (e.g., physiological parameters, device performance parameters, etc.) based on the data. The system may determine certain physiological parameters, for example, that indicate one or more symptoms of a medical condition that requires immediate medical attention or hospitalization. Such physiological parameters can include those related to temperature, patient movement/activity level, heart rate, respiratory rate, blood oxygen saturation, and/or other suitable parameters described herein. Based on these parameters, the system may provide an indication to the patient and/or clinician that the patient has developed or is at risk of developing an illness or is experiencing complications from therapy. The system of the present technology may be especially beneficial for cancer patients undergoing chemotherapy, as chemotherapy has many side effects that could be fatal to the patient if not treated immediately. The vascular access devices, systems, and methods disclosed herein enable early detection of known symptoms, thereby improving patient survival rates and overall quality of life. Additionally or alternatively, the systems of the present technology can be configured to determine one or more device performance parameters. For example, a vascular access device can include a sensing element configured to obtain data characterizing a flow rate within the device and based on the data, the system can determine if the device is occluded and/or the extent of the occlusion. In this manner (and in others), the devices, systems, and methods of the present technology can be configured for monitoring the performance of an implantable device and early detection of any issues with the implantable device.

Additionally, the implantable device may contain data storage and communication technology that not only monitors physiological parameters and logs device communication history, but also contains information about the patient's demographics, diagnoses, treatment history, and POLST (Physician Order for Life Sustaining Treatment) status. In some embodiments, the vascular access device can be configured for wireless communication with an interrogation device or other remote computing device. The interrogation device may also wirelessly recharge a battery of the vascular access device, for example via inductive charging.

To limit power consumption, in some instances it may be beneficial for implantable device to modulate its operation over time depending on certain conditions. This modulation can include limiting wireless data communication with external devices to certain time intervals. Additionally or alternatively, modulation of device operation can include varying which sensing elements are actively collecting data, and/or adjusting a sampling frequency of some or all of the sensing elements. In some examples, during certain time intervals, the implantable device may operate in a low-power standby state in which data is obtained via sensing elements and stored in local data storage, but the data is not wirelessly transmitted (e.g., using Wi-Fi or Bluetooth) to an external device. In response to a modulation signal, the implantable device can “wake up” (e.g., activate) to a fully operational state in which the physiological data (and/or other data) is transmitted to one or more external devices. The modulation signal can originate from a smartphone or other interrogation device (e.g., via near-field communication (NFC) coil, etc.), a controller and/or sensing element carried by the implantable device, and/or another suitable source. After receiving another modulation signal and/or after a predetermined period of time has implantable, the implantable device may return to the low-power standby state. In some embodiments, the implantable device can “go to sleep” (e.g., deactivate or enter a low-power mode) in response to a modulation signal. As such, the overall power consumption of the implantable device can be reduced, and the battery life accordingly extended, by varying operation of the device over time. This can beneficially reduce or eliminate the need for a patient or clinician to actively recharge or even replace an implantable device.

In some embodiments, the implantable device can be configured to transition between two or more states (e.g., from a standby state to an operational state and vice versa, etc.) after a trigger event occurs. The trigger event can comprise a measurement of a physiological parameter that falls above or below a predetermined threshold, a measurement of a physiological parameter that falls outside of a predetermined range, elapsing of a predetermined time, receiving a modulation signal, discontinuation of a modulation signal, and others.

In some embodiments, operation of the implantable device can be modulated based on data collected via the sensing elements. For example, if a patient's physiological parameters deviate from a certain range (e.g., falling outside of a predefined range of acceptable values, deviating more than a certain percentage away from the patient's historical baseline, etc.), the implantable device may modulate its operation by increasing a sampling frequency of one or more sensing elements and/or by initiating wireless data transmission to one or more remote devices. Such transmission can include the physiological parameters detected via the device and/or an alert to the patient, clinician, or other entity. In some embodiments, the implantable device may revert to the low-power or standby state once the patient's physiological parameters return to acceptable levels (e.g., being within the predefined range of acceptable values, moving closer to the patient's historical baseline, etc.). For example, if a patient is developing a fever (e.g., as indicated by temperature measurements, rate of change of temperature measurements, etc. that exceed a predetermined threshold), the frequency of temperature measurements and/or other measurements via the implantable device can increase to obtain data more frequently and/or at a higher resolution. If and/or when the patient's temperature returns to an acceptable value, the frequency of temperature measurements and/or other measurements can be reduced. In some embodiments, if a parameter measured by an implantable device remains within an acceptable range for a predetermined duration of time, the sampling frequency of one or more sensing elements can be decreased until an out-of-range parameter is detected. In some embodiments, the rate of sensing element data collection and/or data transmission can vary based on automated responses to clinical measurements and/or input from a clinician or patient. As a patient has a lower risk of developing a medical condition, the data sampling rate can decrease, and as the patient's risk of developing a medical condition rises, the data sampling rate can increase. For example, if a patient has recently undergone invasive, high-risk medical procedures (e.g., radiation, chemotherapy, surgery, etc.), a clinician can provide input to the patient's implantable device to cause the data sampling rate to be elevated for a period between the medical procedure and the patient's first follow-up appointment.

An implantable device of the present technology can alert the patient and/or clinician in near-real-time when one or more physiological parameters fall outside of expected ranges. This near-real-time monitoring can lead to improved patient outcomes, as the clinician may intervene more rapidly to take suitable measures to address the patient's symptoms. Such interventions may include administering additional therapeutic agents, modifying a dosage of a current therapeutic agent, recommending certain lifestyle changes, or taking any other suitable action. By tailoring the measurement frequency to the recent measurement values, the patient's health can be closely monitored when the detected physiological parameters so warrant, while conserving battery life when the patient's physiological parameters are within normal ranges.

In some embodiments, the interrogation device can take the form of a smartphone, tablet, or other mobile device that can be held in a patient's hand. In operation, the patient may place the interrogation device over the implantable device such that a modulation signal transmitted by the interrogation device (e.g., transmitted via an NFC coil, etc.) can be received at the implantable device. As described in more detail below, the smartphone, tablet, or other mobile device can run software in the form of a dedicated software application (also referred to as “app” herein) configured to receive user input from the patient and present relevant clinical information to the patient. The app can be configured to access additional external data sources, such as remote servers storing patient health records, historical sensor data, input from treating clinicians or other providers, or any other suitable data.

In some embodiments, a patient can use the software application to control and modulate the operational state of the implantable device. For example, the patient can, via the app, cause the mobile device to transmit a modulation signal that in turn causes the implantable device to wake up (i.e., transition from a low-power state to a fully operational state). In some embodiments, the software application can include a graphical representation of data obtained by the implantable device and/or can include input fields for a patient to enter information regarding symptoms, medication, appointments, and other suitable information. In some embodiments, a patient may be limited in the number of interrogations or wake-ups of the implantable device that they may initiate, so as to limit overall power consumption by the implantable device.

In some embodiments, an NFC signal (or other suitable wireless signal) can be used for localization of an implantable device instead of or in addition to modulation of operation of the device. For example, in response to an NFC signal transmitted via an adjacent interrogation device, the implantable device may illuminate one or more LEDs, vibrate, actuate a protruding element, emit any other localization signal, or perform any other action that may facilitate localization of the implantable device. Additionally or alternatively, the NFC signal can be used to guide positioning of the mobile device at a desired position with respect to the implantable device. As one example, a mobile device may display an augmented-reality visualization of the implantable device's location, for example by displaying a camera feed of the patient's body with the detected location of the implantable device indicated by some superimposed graphical representation (e.g., cross-hairs, a dot, an image of an implantable device, etc.). Such localization can be particularly useful in the case of low-profile implantable vascular access devices, which can be difficult to locate visually or via palpation alone.

Various other permutations are possible, as will be appreciated by one of skill in the art. Additionally, although several examples refer to a low-power standby state and a fully operational state, it will be apparent that there may be many intermediate states that have varying power consumption and other characteristics. For example, an implantable device having multiple sensing elements may turn each element on or off independently, and may also modify a sampling or polling schedule for each sensing element. Moreover, the rate or schedule of wireless data transmission (e.g., using Wi-Fi, Bluetooth, or other suitable communication standard) can be modified along a continuum, and need not be toggled only in an on/off manner. In some embodiments, the rate of power consumption can be increased on-demand, for example by increasing the sampling frequency of measurements even above and beyond the normal fully operational levels. This may be performed for a shorter duration in time to achieve a greater resolution of data.

### Monitoring System Overview

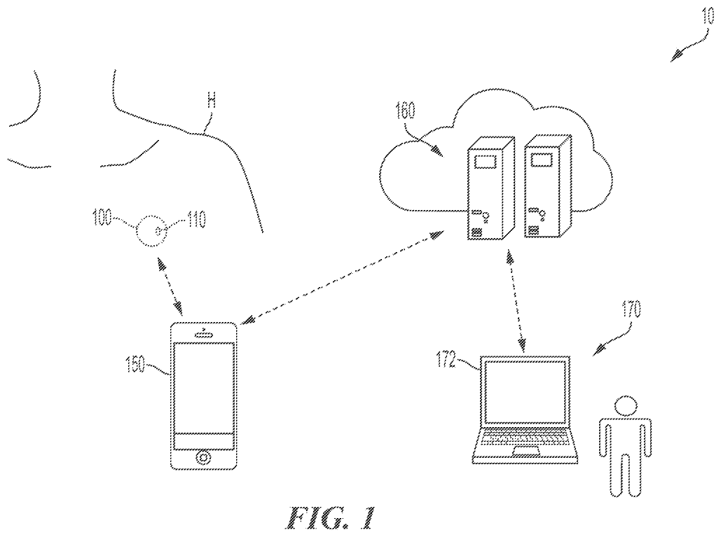

FIG. 1 is a schematic representation of a monitoring system 10 in accordance with the present technology. The system 10 can include a vascular access device 100 (or “device 100”) configured to be implanted within a human patient H, such as at a subcutaneous location along an upper region of the patient's chest. As shown in FIG. 1, the device 100 may include a sensing element 110 configured to obtain physiological measurements that are used by the system 10 to determine one or more physiological parameters indicative of the patient's health. In some embodiments, the system 10 may detect a medical condition (such as sepsis) or associated symptom(s) based on the physiological parameter(s) and provide an indication of the detected condition to the patient, caregiver, and/or medical care team.

As shown schematically in FIG. 1, the device 100 may be configured to communicate wirelessly with a local computing device 150, which can be, for example, a smart device (e.g., a smartphone, a tablet, or other handheld device having a processor and memory), a special-purpose interrogation device, or other suitable device. Communication between the device 100 and the local computing device 150 can be mediated by, for example, near-field communication (NFC), infrared wireless, Bluetooth, ZigBee, Wi-Fi, inductive coupling, capacitive coupling, or any other suitable wireless communication link. The device 100 may transmit data including, for example, physiological measurements obtained via the sensing element 110, patient medical records, device performance metrics (e.g., battery level, error logs, etc.), or any other such data stored by the device 100. In some embodiments, the transmitted data is encrypted or otherwise obfuscated to maintain security during transmission to the local computing device 150. The local computing device 150 may also provide instructions to the vascular access device 100, for example to obtain certain physiological measurements via the sensing element 110, to emit a localization signal, or to perform other functions. In some embodiments, the local computing device 150 may be configured to wirelessly recharge a battery of the device 100, for example via inductive charging.

The system 10 may further include first remote computing device(s) 160 (or server(s)), and the local computing device 150 may in turn be in communication with first remote computing device(s) 160 over a wired or wireless communications link (e.g., the Internet, public and private intranet, a local or extended Wi-Fi network, cell towers, the plain old telephone system (POTS), etc.). The first remote computing device(s) 160 may include one or more own processor(s) and memory. The memory may be a tangible, non-transitory computer-readable medium configured to store instructions executable by the processor(s). The memory may also be configured to function as a remote database, i.e., the memory may be configured to permanently or temporarily store data received from the local computing device 150 (such as one or more physiological measurements or parameters and/or other patient information).

In some embodiments, the first remote computing device(s) 160 can additionally or alternatively include, for example, server computers associated with a hospital, a medical provider, medical records database, insurance company, or other entity charged with securely storing patient data and/or device data. At a remote location 170 (e.g., a hospital, clinic, insurance office, medical records database, operator's home, etc.), an operator may access the data via a second remote computing device 172, which can be, for example a personal computer, smart device (e.g., a smartphone, a tablet, or other handheld device having a processor and memory), or other suitable device. The operator may access the data, for example, via a web-based application. In some embodiments, the obfuscated data provided by the device 100 can be de-obfuscated (e.g., unencrypted) at the remote location 170.

In some embodiments, the device 100 may communicate with remote computing devices 160 and/or 172 without the intermediation of the local computing device 150. For example, the vascular access device 100 may be connected via Wi-Fi or other wireless communications link to a network such as the Internet. In other embodiments, the device 100 may be in communication only with the local computing device 150, which in turn is in communication with remote computing devices 160 and/or 172.

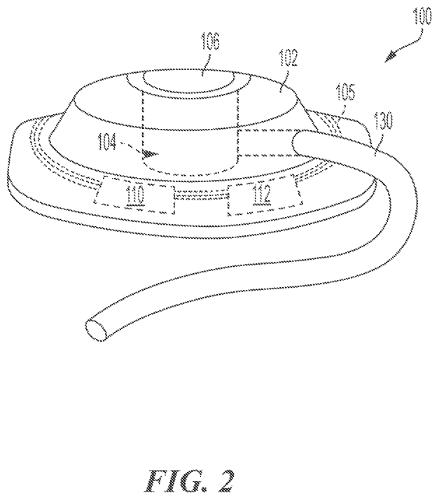

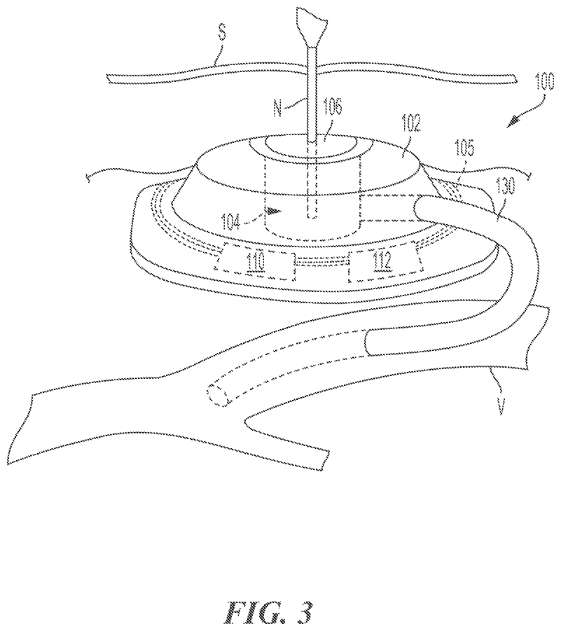

FIG. 2 shows an example of a vascular access device 100 (or “device 100”) configured for use with the system 10 of the present technology. As shown in FIG. 2, the device 100 comprises a housing 102 configured to be implanted within a human patient, a fluid reservoir 104 contained within the housing 102, and a septum 106 adjacent the reservoir 104 and configured to receive a needle therethrough for delivery of a fluid (such as a therapeutic or diagnostic agent) to the reservoir 104 (as described in greater detail below with respect to FIG. 3). The housing 102 may be made of a biocompatible plastic, metal, ceramic, medical grade silicone, or other material that provides sufficient rigidity and strength to prevent needle puncture. The septum 106 can be, for example, a self-sealing membrane made of silicone or other deformable, self-sealing, biocompatible material. In some embodiments, the device 100 may include a catheter 130 that extends distally from the housing 102 and is in fluid communication with the reservoir 104. For example, the catheter 130 can be configured to mate with an outlet port of the device 100 via a barb connector or other suitable mechanical connection. The catheter 130 may be a single or multi-lumen catheter. In some embodiments, the device 100 includes multiple separate catheters.

As shown in FIG. 3, in operation the device 100 is implanted in a patient beneath the skin S, for example in a small pocket created in the upper chest wall just inferior to the clavicle. The catheter 130, which is in fluid communication with the reservoir 104, is inserted into a blood vessel V, for example the internal jugular vein or the subclavian vein with the tip resting in the superior vena cava or the right atrium. A clinician inserts a needle N (e.g., a non-coring or Huber-type needle) through the skin S, through the self-sealing septum 106, and into the fluid reservoir 104. To introduce fluid (e.g., medication) into the patient's blood vessel V, the clinician may advance fluid through the needle N, which then flows through the reservoir 104, the catheter 130, and into the vessel V, or the physician may advance fluid through the needle to fill the reservoir for postponed delivery into the vessel V. To remove fluid from the vessel V (e.g., to aspirate blood from the vessel V for testing), the clinician can apply suction via the needle N, thereby withdrawing fluid (e.g., blood) from the vessel V into the catheter 130, into the fluid reservoir 104, and into the needle N. When the procedure is completed, the clinician removes the needle N, the self-sealing septum 106 resumes a closed configuration, and the device 100 may remain in place beneath the patient's skin S.

Referring again to FIG. 2, the device 100 includes a sensing element 110 carried by the housing 102. The sensing element 110 can be configured to obtain data characterizing a physiological parameter of a patient, a performance parameter of the device 100, and/or other information related to treatment and/or care of the patient. Although a single sensing element 110 is illustrated for clarity, the device 100 may include a plurality of sensing elements 110 disposed within coupled to, or otherwise carried by the housing 102. In some embodiments, one or more such sensing elements 110 may be disposed on separate structural components that are separated from the housing 102. As used herein, the term “sensing element” may refer to a single sensor or a plurality of discrete, separate sensors.

The device 100 may include at least one controller 112 communicatively coupled to the sensing element 110. The controller 112 may include one or more processors, software components, and memory (not shown). In some examples, the one or more processors include one or more computing components configured to process the data obtained by the sensing element 110 according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the sensing element 110 to obtain data characterizing a patient's health, performance of the device, treatment status, and/or other parameters for enhancing patient care. In another example, the functions may involve processing physiological data to determine one or more physiological parameters and/or provide an indication to the patient and/or clinician of one or more symptoms or medical conditions associated with the determined physiological parameters.

The controller 112 may also include a data communications unit configured to securely transmit data between the device 100 and external computing devices (e.g., local computing device 150, remote computing devices 160 and 172, etc.). In some embodiments, the controller 112 includes a localization unit configured to emit a localization signal (e.g., lights that transilluminate a patient's skin, vibration, a magnetic field, etc.) to aid a clinician in localizing the device 100 when implanted within a patient. The controller 112 can also include a wireless charging unit (such as a coil) configured to recharge a battery (not shown) of the device 100 when in the presence of an interrogation device (e.g., local device 150 or another suitable device).

The system 10 may be configured to continuously and/or periodically obtain measurements via the sensing element 110 in communication with the device 100. The sensing element 110 may be carried by the housing 102 and/or the catheter 130, and/or may include a sensing component separate from the housing 102 and catheter 130 but physically or communicatively coupled to the housing 102 and/or catheter 130. The sensing element 110 may be implanted at the same location as the device 100 or at a different location, or may be positioned on the patient at an exterior location (e.g., on the patient's skin). The sensing element 110 may be permanently coupled to the device 100, or may be configured to temporarily couple to the device 100.

In some embodiments, the sensing element 110 is built into the housing 102 such that only a portion of the sensing element 110 is exposed to the local physiological environment when the device 100 is implanted. For example, the sensing element 110 may comprise one or more electrodes having an external portion positioned at an exterior surface of the housing 102 and an internal portion positioned within the housing 102 and wired to the controller 112. In some embodiments, the sensing element 110 may comprise one or more electrodes having an internal portion positioned at an interior surface of the housing 102 at the interface with the port reservoir 104 or junction of the reservoir 104 and the catheter 130, or extending into the catheter 130.

In some embodiments, the sensing element 110 may be completely contained within the housing 102. For example, the sensing element 110 may comprise one or more pulse oximeters enclosed by the housing 102 and positioned adjacent a window in the housing 102 through which light emitted from the pulse oximeter may pass to an external location, and back through which light reflected from the external location may pass for detection by a photodiode of the pulse oximeter. In such embodiments the window may be, for example, a sapphire window that is brazed into place within an exterior wall of the housing 102.

In at least some embodiments, the sensing element 110 can be contained within a discrete and separate enclosure that is coupled to (e.g., attached directly, via a tether or intervening structure, etc.) to the housing 102.

The sensing element 110 may comprise at least one sensor completely enclosed by the housing 102 and at least one sensor that is partially or completely positioned at an external location, whether directly on the housing 102 and/or catheter 130 or separated from the housing 102 and/or catheter 130 (but still physically coupled to the housing 102 and/or catheter 130 via a wired connection, for example). In some embodiments, at least a portion of the sensing element 110 is positioned at and/or exposed to an interior region of the reservoir 104.

In some embodiments, the sensing element 110 may include a separate controller (not shown) that comprises one or more processors and/or software components. In such embodiments, the sensing element 110 may process at least some of the measurements characterized by data obtained by the sensing element 110 to determine one or more parameters associated with the data, and then transmit those parameters to the controller 112 of the device 100 (with or without the underlying data). In some examples, the sensing element 110 may only partially process at least some of the measurements before transmitting the data to the controller 112. In such embodiments, the controller 112 may further process the received data to determine one or more parameters. The local computing device 150 and/or the remote computing devices 160, 172 may also process some or all of the measurements obtained by the sensing element 110 and/or parameters determined by the sensing element 110 and/or the controller 112.

According to some aspects of the technology, the sensing element 110 may include memory. The memory may be a non-transitory computer-readable medium configured to permanently and/or temporarily store the measurements obtained by the sensing element 110. In those embodiments where the sensing element 110 includes its own processor(s), the memory may be a tangible, non-transitory computer-readable medium configured to store instructions executable by the processor(s).

In some embodiments, the sensing element(s) 110 and/or controller 112 may identify, monitor, and communicate patient information by electromagnetic, acoustic, motion, optical, thermal, or biochemical sensing elements or means. The sensing element(s) 110 may include, for example, one or more temperature sensing elements (e.g., one or more thermocouples, one or more digital temperature sensors, one or more thermistors or other type of resistance temperature detector, etc.), one or more impedance sensing elements (e.g., one or more electrodes), one or more pressure sensing elements, one or more optical sensing elements, one or more flow sensing elements (e.g., a Doppler velocity sensing element, an ultrasonic flow meter, etc.), one or more ultrasonic sensing elements, one or more pulse oximeters, one or more chemical sensing elements, one or more movement sensing elements (e.g., one or more accelerometers), one or more pH sensing elements, an electrocardiogram (“ECG” or “EKG”) unit, one or more electrochemical sensing elements, one or more hemodynamic sensing elements, and/or other suitable sensing devices.

The sensing element 110 may comprise one or more electromagnetic sensing elements configured to measure and/or detect, for example, impedance, voltage, current, or magnetic field sensing capability with a wire, wires, wire bundle, magnetic node, and/or array of nodes. The sensing element 110 may comprise one or more acoustic sensing elements configured to measure and/or detect, for example, sound frequency, within human auditory range or below or above frequencies of human auditory range, beat or pulse pattern, tonal pitch melody, and/or song. The sensing element 110 may comprise one or more motion sensing elements configured to measure and/or detect, for example, vibration, movement pulse, pattern or rhythm of movement, intensity of movement, and/or speed of movement. Motion communication may occur by a recognizable response to a signal. This response may be by vibration, pulse, movement pattern, direction, acceleration, or rate of movement. Motion communication may also be by lack of response, in which case a physical signal, vibration, or bump to the environment yields a motion response in the surrounding tissue that can be distinguished from the motion response of the sensing element 110. Motion communication may also be by characteristic input signal and responding resonance. The sensing element 110 may comprise one or more optical sensing elements which may include, for example, illuminating light wavelength, light intensity, on/off light pulse frequency, on/off light pulse pattern, passive glow or active glow when illuminated with special light such as UV or “black light”, or display of recognizable shapes or characters. It also includes characterization by spectroscopy, interferometry, response to infrared illumination, and/or optical coherence tomography. The sensing element 110 may comprise one or more thermal sensing elements configured to measure and/or detect, for example, device 100 temperature relative to surrounding environment, the temperature of the device 100 (or portion thereof), the temperature of the environment surrounding the device 100 and/or sensing element 110, or differential rate of the device temperature change relative to surroundings when the device environment is heated or cooled by external means. The sensing element 110 may comprise one or more biochemical devices which may include, for example, the use of a catheter, a tubule, wicking paper, or wicking fiber to enable micro-fluidic transport of bodily fluid for sensing of protein, RNA, DNA, antigen, and/or virus with a micro-array chip.

In some aspects of the technology, the controller 112 and/or sensing element 110 may be configured to detect and/or measure the concentration of blood constituents, such as sodium, potassium, chloride, bicarbonate, creatinine, blood urea nitrogen, calcium, magnesium, and phosphorus. The system 10 and/or the sensing element 110 may be configured to evaluate liver function (e.g., by evaluation and/or detection of AST, ALT, alkaline phosphatase, gamma glutamyl transferase, troponin, etc.), heart function (e.g., by evaluation and/or detection of troponin), coagulation (e.g., via determination of prothrombin time (PT), partial thromboplastin time (PTT), and international normalized ratio (INR)), and/or blood counts (e.g., hemoglobin or hematocrit, white blood cell levels with differential, and platelets). In some embodiments, the system 10 and/or the sensing element 110 may be configured to detect and/or measure circulating tumor cells, circulating tumor DNA, circulating RNA, multigene sequencing of germ line or tumor DNA, markers of inflammation such as cytokines, C reactive protein, erythrocyte sedimentation rate, tumor markers (PSA, beta-HCG, AFP, LDH, CA 125, CA 19-9, CEA, etc.), and others.

The system 10 may be configured to determine one or more physiological parameters based on the physiological measurements and/or one or more other physiological parameter(s). For example, the system 10 may be configured to determine physiological parameters such as heart rate, temperature, blood pressure (e.g., systolic blood pressure, diastolic blood pressure, mean blood pressure), blood flow rate, blood velocity, pulse wave speed, volumetric flow rate, reflected pressure wave amplitude, augmentation index, flow reserve, resistance reserve, resistive index, capacitance reserve, any blood cellular level, count, or other cellular measurement (e.g., hematocit) or any blood chemistry (e.g., blood glucose, potassium, etc, and the like), heart rhythm, electrocardiogram (ECG) tracings, body fat percentage, activity level, body movement, falls, gait analysis, seizure activity, blood glucose levels, drug/medication levels, blood gas constituents and blood gas levels (e.g., oxygen, carbon dioxide, etc.), lactate levels, hormone levels (such as cortisol, thyroid hormone (T4, T3, free T4, free T3), TSH, ACTH, parathyroid hormone), and/or any correlates and/or derivatives of the foregoing measurements and parameters (e.g., raw data values, including voltages and/or other directly measured values). In some embodiments, one or more of the physiological measurements can be utilized or characterized as a physiological parameter without any additional processing by the system 10.

Additionally or alternatively, the sensing element 110 can be configured to obtain data characterizing a parameter associated with performance of the device, treatment of the patient, etc. For example, the sensing element 110 can be configured to obtain data characterizing a flow rate parameter within the catheter 130 and/or the reservoir 104, a pressure within the catheter 130 and/or the reservoir 104, a temperature of one or more portions of the device 100, a presence and/or position of an object (e.g., a needle, fluid, a clot, etc.) within the reservoir 104 and/or catheter 130, information encoded by machine-readable indicia, etc.

The system 10 may also determine and/or monitor derivatives of any of the foregoing parameters (e.g., physiological parameters, device performance parameters, treatment parameters, identity parameters, etc.), such as a rate of change of a particular parameter, a change in a particular parameter over a particular time frame, etc. As but a few examples, the system 10 may be configured to determine as temperature over a specified time, a maximum temperature, a maximum average temperature, a minimum temperature, a temperature at a predetermined or calculated time relative to a predetermined or calculated temperature, an average temperature over a specified time, a maximum blood flow, a minimum blood flow, a blood flow at a predetermined or calculated time relative to a predetermined or calculated blood flow, an average blood flow over time, a maximum impedance, a minimum impedance, an impedance at a predetermined or calculated time relative to a predetermined or calculated impedance, a change in impedance over a specified time, a change in impedance relative to a change in temperature over a specified time, a change in heart rate over time, a change in respiratory rate over time, activity level over a specified time and/or at a specified time of day, and other suitable derivatives.

Measurements may be obtained continuously or periodically at one or more predetermined times, ranges of times, calculated times, and/or times when or relative to when a measured event occurs. Likewise, parameters may be determined continuously or periodically at one or more predetermined times, ranges of times, calculated times, and/or times when or relative to when a measured event occurs.

Based on the determined parameters, the system 10 of the present technology is configured to provide an indication of the patient's health, the performance and/or health of device, and/or the status of a treatment to the patient and/or a clinician. For example, the controller 112 may compare one or more of the physiological parameters to a predetermined threshold or range and, based on the comparison, provide an indication of the patient's health. For instance, if the determined physiological parameter(s) is above or below the predetermined threshold or outside of the predetermined range, the system 10 may provide an indication that the patient is at risk of, or has already developed, a medical condition characterized by symptoms associated with the determined physiological parameters. As used herein, a “predetermined range” refers to a set range of values, and “outside of a/the predetermined range” refers to (a) a measured or calculated range of values that only partially overlap the predetermined range or do not overlap any portion of a predetermined range of values. As used herein, a “predetermined threshold” refers to a single value or range of values, and a parameter that is “outside” of “a predetermined threshold” refers to a situation where the parameter is (a) a measured or calculated value that exceeds or fails to meet a predetermined value, (b) a measured or calculated value that falls outside of a predetermined range of values, (c) a measured or calculated range of values that only partially overlaps a predetermined range of values or does not overlap any portion of a predetermined range of values, or (d) a measured or calculated range of values where none of the values overlap with a predetermined value.

Predetermined parameter thresholds and/or ranges can be empirically determined to create a look-up table. Look-up table values can be empirically determined, for example, based on clinical studies and/or known healthy or normal values or ranges of values. The predetermined threshold may additionally or alternatively be based on a particular patient's baseline physiological parameters, a particular device's baseline performance parameters, etc.

Medical conditions detected and/or indicated by the system 10 may include, for example, sepsis, pulmonary embolism, metastatic spinal cord compression, anemia, dehydration/volume depletion, vomiting, pneumonia, congestive heart failure, performance status, arrhythmia, neutropenic fever, acute myocardial infarction, pain, opioid toxicity, nicotine or other drug addiction or dependency, hyperglycemic/diabetic ketoacidosis, hypoglycemia, hyperkalemia, hypercalcemia, hyponatremia, one or more brain metastases, superior vena cava syndrome, gastrointestinal hemorrhage, immunotherapy-induced or radiation pneumonitis, immunotherapy-induced colitis, diarrhea, cerebrovascular accident, stroke, pathological fracture, hemoptysis, hematemesis, medication-induced QT prolongation, heart block, tumor lysis syndrome, sickle cell anemia crisis, gastroparesis/cyclic vomiting syndrome, hemophilia, cystic fibrosis, chronic pain, and/or seizure. Any of the systems and/or devices disclosed herein may be used to monitor a patient for any of the foregoing medical conditions.

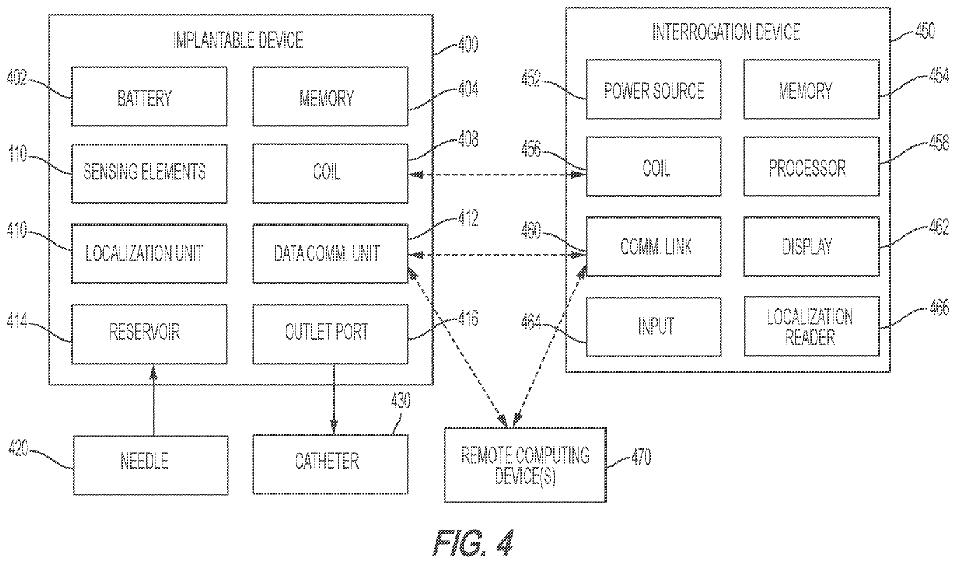

FIG. 4 is a schematic block diagram of an environment for communication between an implantable device 400, an interrogation device 450, and one or more remote computing devices 470. The implantable device 400 can be a vascular access device (e.g., the device 100 described above with respect to FIGS. 1-3). In some embodiments, the implantable device 400 can be another implantable medical device, for example, a pacemaker, implantable cardioverter/defibrillator (ICD), deep brain stimulator, insulin pump, infusion port, orthopedic device, pulmonary artery pressure monitor, or any other implantable medical device with electronic sensing components.

The interrogation device 450 can be, for example, a handheld device configured to communicate wirelessly with the implantable device 400 when the device 400 is implanted within a patient. This communication can be carried out using a short-range connection (e.g., near-field communication (NFC), infrared wireless, Bluetooth, ZigBee, Wi-Fi, inductive coupling, or capacitive coupling) or other suitable wireless communication link. In various embodiments, the implantable device 400 and/or the interrogation device 450 can communicate with one or more remote computing devices 470, for example over a network connection such as the Internet.

In the illustrated embodiment, the implantable device 400 can include a battery 402 (e.g., a rechargeable battery or other power source), and memory 404. The memory 404 can include read-only memory (ROM) and random access memory (RAM) or other storage devices such as SSDs that store the executable applications, test software, databases and other software required to, for example, implement the various routines described herein, control device components, communicate and exchange data and information with remote computers and other devices, etc. The implantable device 400 can include a number of electronic elements (e.g., the memory 404, sensing elements 110, coil 408, the localization unit 410, and/or the data communications unit 412). Some or all of these elements can include one or more processors, analog-to-digital converters, data storage devices, wireless communication antennas, and other associated elements. Some or all of these elements can be electronically coupled to or carried by a printed circuit board (e.g., a rigid or flexible PCB) or other suitable substrate. In some embodiments, software or firmware stored in the memory 404 or on a microprocessor unit can be configured to optimize data collection, communication, localization, and battery life of the device 400.

The implantable device 400 includes sensing elements 110 configured to obtain one or more measurements (e.g., physiological, device performance, etc.) while implanted within the body. As described above with respect to FIGS. 1-3, the sensing elements 110 can be configured to obtain any number of different measurements and/or one or more other parameters. For the example, the sensing elements 110 may be configured to determine physiological parameters such as heart rate, temperature, blood pressure (e.g., systolic blood pressure, diastolic blood pressure, mean blood pressure), blood flow rate, blood velocity, pulse wave speed, volumetric flow rate, reflected pressure wave amplitude, augmentation index, flow reserve, resistance reserve, resistive index, capacitance reserve, hematocrit, heart rhythm, electrocardiogram (ECG) tracings, body fat percentage, activity level, body movement, falls, gait analysis, seizure activity, blood glucose levels, drug/medication levels, blood gas constituents and blood gas levels (e.g., oxygen, carbon dioxide, etc.), lactate levels, hormone levels (such as cortisol, thyroid hormone (T4, T3, free T4, free T3), TSH, ACTH, parathyroid hormone), and/or any correlates and/or derivatives of the foregoing measurements and parameters (e.g., raw data values, including voltages and/or other directly measured values). Additionally or alternatively, the sensing elements 110 may be configured to determine device performance parameters such as a temperature of the device, a pressure within the device, a flow rate within the device, etc.

The device 400 can also include a coil 408 (e.g., an antenna), for example a length of electrically conductive wire or other material that is wrapped to form a circular coil or other shape. In some embodiments, the coil 408 can be a conductive wire that encircles the reservoir 414 of the device 400. The coil 408 can be electrically coupled to the battery 402 such that electrical energy received via the coil 408 can be used to recharge the battery 402. In some embodiments, the coil 408 can also be electrically coupled to the localization unit 410 such that electrical energy received via the coil 408 causes the localization unit 410 to emit a localization signal. Additionally or alternatively, the coil 408 can be electrically coupled to the data communications unit 412 such that electrical energy received via the coil 408 causes the data communications unit 412 to perform certain actions, for example securely transmitting data to the interrogation device 450. The coil 408 can be inductively coupled to a coil 456 of the interrogation device 450 to wirelessly receive electrical energy from the coil 456. In some embodiments, the wireless energy is transmitted via capacitive coupling rather than inductive coupling.

With continued reference to FIG. 4, the implantable device 400 can further include a localization unit 410. The localization unit 410 can include an emitter configured to emit a localization signal and/or a controller (e.g., a central processing unit, digital signal processor, application-specific integrated circuit, or any other logic processing unit) that reads instructions from the memory 404 to perform suitable operations or performs operations based on firmware stored on a microprocessor unit. The localization unit 410 can be configured to emit one or more localization signals from the implantable device 400 to aid a clinician in identifying the location of the device 400 when implanted within a patient. As noted previously, in some embodiments the localization unit 410 is configured to emit a localization signal in response to detecting the presence of the interrogation device 450. For example, the coil 456 of the interrogation device 450 can be driven with an alternating current suitable to induce a current in the coil 408 of the implantable device 400 when the two devices are held in proximity to one another such that the coil 456 of the interrogation device 450 and the coil 408 of the implantable device 400 are inductively coupled. The induced electrical current in the coil 408 of the implantable device can, in turn, cause the localization unit 410 to emit a localization signal. In some embodiments, the interrogation device 450 can include a localization reader 466 that is configured to read, detect, or otherwise identify a localization signal emitted by the localization unit 410 of the implantable device 400. In other embodiments, the localization reader 466 can be omitted from the interrogation device 450, and a clinician may directly observe the localization signal emitted by the localization unit 410.

In various embodiments, the localization unit 410 can take a variety of forms, having different configurations of emitters configured to emit different localization signals, and a corresponding localization reader 466 of the interrogation device 450 can be configured to read or detect the particular localization signal emitted by the localization unit 410. In each of the following examples, in some embodiments the interrogation device 450 may not include a localization reader 466, and instead the localization signal emitted from the localization unit 410 of the implantable device 400 may be read, identified, observed, or detected either directly by the user (e.g., a clinician, etc.) or by using another suitable instrument. In one example, the localization unit 410 can include one or more light sources disposed about the device 100, and the localization signal can include the emission of light from the light sources. The emitted light can be configured to transilluminate the skin to indicate a location of the implantable device 400 to a clinician. In this instance, the localization reader 466 can include a light sensor or array of sensors configured to identify the lights transilluminating the patient's skin.

In further examples, the localization signal may take a variety of other forms. In some embodiments, the localization unit 410 includes a speaker configured to emit an audible sound as the localization signal, and the localization reader 466 includes a microphone or other device configured to detect the emitted sound and to localize its source. In some embodiments, the localization unit 410 includes one or more magnets (e.g., permanent magnets or electromagnets), and the localization signal includes the magnetic field generated by the magnets. For example, a plurality of magnets may be disposed around a reservoir of the implantable device 400, and the magnetic field generated by these magnets may be detected by the localization reader 466 of the interrogation device 450 in a manner that indicates the location of the reservoir or other aspect of the implantable device 400. In some embodiments, the localization unit 410 includes a radiofrequency transmitter, and the localization signal includes a radiofrequency signal that can be detected by the interrogation device 450. In this instance, the localization reader 466 can be an antenna or other device configured to detect the signature radiofrequency signal emitted by the interrogation device and to localize the source of the signals. In some embodiments, the localization unit 410 includes an actuator configured to move or vibrate certain elements to serve as the localization signal. In some embodiments, the localization unit 410 includes one or more ultrasound transducers, and the emitted ultrasound serves as a localization signal to be detected by the localization reader 466 interrogation device 450. In some embodiments, the localization unit 410 includes at least one moveable member that can create a temporary protrusion raising from an upper surface of the implantable device such that the protrusion can be palpated by a clinician to localize the device 400. In some embodiments, the localization unit 410 includes a radioisotope and the localization signal comprises the electromagnetic radiation emitted by the radioisotope. For example, the localization unit 410 may include a retractable shield that absorbs radiation emitted by the radioisotope. To emit the localization signal, the localization unit 410 can cause the shield to be retracted, thereby allowing the radiation emitted by the radioisotope to escape the device 400 to be detected by the localization reader 466 of the interrogation device 450. In some embodiments, the localization unit 410 includes a heating element and the localization signal is the increased heat signature radiating from the heating element. The increased temperature can be detected via a thermal camera, temperature sensor, or other suitable element of the localization reader 466. In some embodiments, the localization unit 410 can cause the data communications unit 412 to send patient data or other identifying data to serve as a localization signal. The localization reader 466 may identify the source of the signal by triangulating its position to identify the location of the device 400.

In some embodiments, the localization unit 410 determines whether to emit a localization signal based on a characteristic of the interrogation device 450 that induces a current in the coil 408 of the implantable device 400. For example, the localization unit 410 may assess a characteristic such as a field intensity threshold of electrical energy received from the interrogation device 450, a frequency of the electrical energy received from the interrogation device 450, etc. These characteristics can aid in discriminating between a trusted interrogation device (i.e., an interrogation device suitable for pairing) and a non-trusted interrogation device (i.e., an interrogation device unsuitable for pairing), such that only pre-authorized interrogation devices 450 are able to cause the localization unit 410 to emit a localization signal.

The implantable device 400 also includes a data communications unit 412 that is configured to communicate wirelessly with the interrogation device 450 (via communications link 460). Communication between the data communications unit 412 and the interrogation device 450 can be mediated by, for example, near-field communication (NFC), infrared wireless, Bluetooth, ZigBee, Wi-Fi, inductive coupling, capacitive coupling, or any other suitable wireless communication link. The data communications unit 412 may transmit data including, for example, physiological measurements obtained via the sensing elements 110, patient medical records, device performance metrics (e.g., battery level, error logs, etc.), or any other such data obtained and/or stored by the implantable device 400. The data communications unit 412 may also receive data from the interrogation device 450 (via the communications link 460). For example, the data communications unit 412 may receive instructions to obtain certain physiological measurements via the sensing elements 110, to emit a localization signal via the localization unit 410, or to perform other functions.

In some embodiments, the data communications unit 412 determines whether to transmit data to the interrogation device 450 and/or remote computing devices 470 based on a characteristic of the interrogation device 450 that induces a current in the coil 408 of the implantable device 400. For example, the data communications unit 412 may assess a characteristic such as a field intensity threshold of electrical energy received from the interrogation device 450, a frequency of the electrical energy received from the interrogation device 450, etc. These characteristics can aid in discriminating between a trusted interrogation device (i.e., an interrogation device suitable for pairing) and a non-trusted interrogation device (i.e., an interrogation device unsuitable for pairing), such that only pre-authorized interrogation devices 450 are able to cause the data communications unit 412 to transmit data to the interrogation device 450.

In at least some embodiments, the implantable device 400 may communicate with the interrogation device 450 in a manner that causes the interrogation device 450 to transition to an active state (e.g., to “wake up” the interrogation device). Such a wake-up signal may be initiated, for example, upon the detection of one or more physiological parameters that exceed a predetermined threshold, or based upon any other suitable trigger condition. In some embodiments, an alert, alarm, or other notification can be generated and transmitted to the patient and/or provider. Additionally or alternatively, following the trigger, the implantable device 400 may increase the sampling rate for a given period of time.

In some embodiments, the implantable device 400 may also be in communication with the remote computing device(s) 470 over a wireless communications link (e.g., the Internet, public and private intranet, a local or extended Wi-Fi network, cell towers, etc.). The remote computing device(s) 470 can be, for example, server computers associated with a hospital, medical provider, medical records database, insurance company, or other entity charged with securely storing patient data and/or device data. The data transmitted to the interrogation device 450 and/or the remote computing device(s) by the implantable device 400 can be obfuscated and/or encrypted. In some embodiments, the obfuscated data provided by the data communications unit 412 can be de-obfuscated (e.g., unencrypted) at a remote location. In some embodiments, the implantable device 400 may be in direct communication only with the interrogation device 450, which in turn is in communication with remote computing device(s) 470.

In some embodiments, the interrogation device 450 and/or one or more of the remote computing device(s) 470 can comprise a wearable device, another implantable device, or another suitable device. For example, an implantable device 400 of the present technology can be configured to communicate with a smartwatch, a fitness tracker, a heart rate monitor, a pacemaker, an insulin pump, etc. Such additional devices can be configured to obtain data and/or communicate such data to the implantable device 400 and/or receive data from the implantable device 400.

The implantable device 400 can also include a reservoir 414 that is in fluid communication with an outlet port 416. In use, a needle 420 can be removably inserted into the fluid reservoir 414, and a catheter 430 can be fluidically coupled to the reservoir 414, thereby establishing a fluid path between the needle 420 and the catheter 430 for introduction of fluid (e.g., medication) or withdrawal of fluid (e.g., aspiration of blood for testing). In some embodiments, the implantable device 400 may omit the reservoir or outlet port, for example in the case of pacemakers, deep brain stimulators, or other implantable devices that do not require delivery or extraction of fluids. In some embodiments, the implantable device 400 may include other elements that serve additional functions—for example a pacemaker can include a pacemaking unit configured to deliver current to cardiac leads, etc.

As noted previously, the implantable device 400 is configured to communicate wirelessly with the interrogation device 450. The interrogation device 450 can be, for example, a special-purpose interrogation device, a smartphone (with or without associated accessory hardware such as a conductive coil), tablet, or other suitable computing device configured to communicate with the implantable device 400. The interrogation device 450 can include a power source 452 (e.g., a battery or wired connection for external power), a memory 454, and a processor 458. In some embodiments, the interrogation device 450 can also include a display 462 (e.g., an electronic screen) configured to display information visually to a user, and an input 464 (e.g., buttons, a touch-screen input, etc.) configured to receive user input.

The interrogation device 450 can also include a coil 456 (e.g., an NFC coil, etc.) configured to inductively couple with the coil 408 of the implantable device. For example, when the implantable device 400 and the interrogation device 450 are placed in proximity to one another, an alternating current driven through the coil 456 of the interrogation device 450 creates an alternating magnetic field that, in turn, induces an electrical current in the coil 408 of the implantable device. This induced current in the coil 408 can be used for communication, and/or may also be used to recharge the battery 402, cause the localization unit 410 to emit a localization signal, and/or to cause the data communications unit 412 to transmit data to or receive data from the interrogation device 450.

The interrogation device 450 can include a communications link 460 configured to communicate with the data communications unit 412 of the implantable device 400 and/or to communicate with the remote computing device(s) 470. The communications link 460 can include a wired connection (e.g., an Ethernet port, cable modem, FireWire cable, Lightning connector, USB port, etc.) or a wireless connection (e.g., including a Wi-Fi access point, Bluetooth transceiver, near-field communication (NFC) device, and/or wireless modem or cellular radio utilizing GSM, CDMA, 3G, 4G, and/or 5G technologies).

In some embodiments, the interrogation device 450 can include a localization reader 466 that is configured to read, identify, or detect localization signals emitted via the localization unit 410 of the implantable device 400. In various embodiments, the localization reader 466 can include a light sensor or array, a microphone or array of microphones, a magnetic field probe (e.g., an array of Hall effect sensors), an antenna or other radiofrequency receiver, an ultrasound receiver, an electromagnetic sensor, a temperature sensor, or any other transducer or sensor configured to detect, identify, or read a localization signal emitted via the localization unit 410 of the implantable device 400.

### Selected Examples of Conserving Battery Power of an Implantable Device

Operation of one or more electronic components of an implantable device disclosed herein can be modulated to enhance a lifetime of the device, a performance of the device, etc. In some embodiments, operation of one or more electronic components of an implantable device can be modulated to maintain and/or preserve battery life of the implantable device. For example, operation of the data communications unit, one or more sensing elements, a controller, etc. can be limited to certain time intervals and/or can be responsive to certain conditions. In some examples, data transmission via Bluetooth Low Energy or other such communication standard consumes significantly more power than other functions of the implantable device. As such, rather than constantly transmitting data to an interrogation device, the data communications unit may be configured to transmit data only in accordance with certain predetermined conditions. For example, the data communications unit can be configured to transmit data only after a modulation signal has been received by the implantable device. In some embodiments, an interrogation device can be configured to produce an interrogation signal that serves as the modulation signal. Moreover, collection of data at high frequencies, from multiple sensing elements, and/or at multiple timepoints can require substantial power consumption. Accordingly, a controller of an implantable device can be configured to cause one or more sensing elements of the implantable device to obtain data only after receiving a modulation signal and/or can be configured to modulate a parameter of the data collection (e.g., a frequency, a sample size, etc.) by one or more sensing elements after receiving the modulation signal.

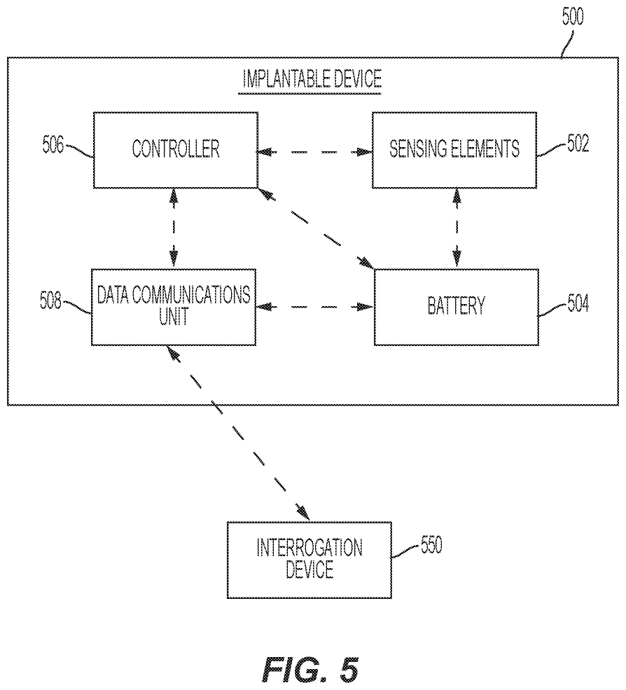

FIG. 5 is a is a schematic block diagram of an environment for communication between an implantable device 500 and an interrogation device 550 (e.g., a handheld device such as a smartphone, a special-purpose interrogation device, an external computing device, etc.). The implantable device 500 includes one or more sensing elements 502 and a battery 504. The one or more sensing elements 502 can be similar to any of the sensing elements disclosed herein. For example, the one or more sensing elements 502 can be configured to obtain physiological data and/or device performance data. The device 500 additionally includes a controller 506 (e.g., an ultra-low power microcontroller) and a data communications unit 508. In some embodiments, the data communications unit 508 includes an NFC transceiver and/or a Bluetooth transceiver. Although NFC and Bluetooth transceivers are referred to here as examples, in other embodiments other wireless communication standards may be used in lieu of NFC and/or Bluetooth. In operation, a modulation signal transmitted via the interrogation device 550 can be received over a first wireless communication link that consumes relatively low power. The first wireless communication link can comprise, for example, electromagnetic coupling (e.g., NFC, etc.). In response to receiving a modulation signal, the implantable device 500 can modulate its operation, for example by “waking up” and transmitting data from the data communications unit 508 via a second wireless communication link. In some embodiments, transmitting data via the second wireless communication link consumes more power than receiving the modulation signal via the first wireless communication link. Such second wireless communication link may comprise direct radio transmission (e.g., WiFi, Bluetooth Low Energy, etc.). By utilizing two wireless communications links in this manner, the overall power consumption of the implantable device 500 can be reduced, and the battery life can be extended. Although electromagnetic coupling (e.g., NFC) and direct radio transmission (e.g., WiFi, Bluetooth, etc.) are disclosed herein, various other options are possible for the different wireless links, both with respect to a lower power link and a higher power link.

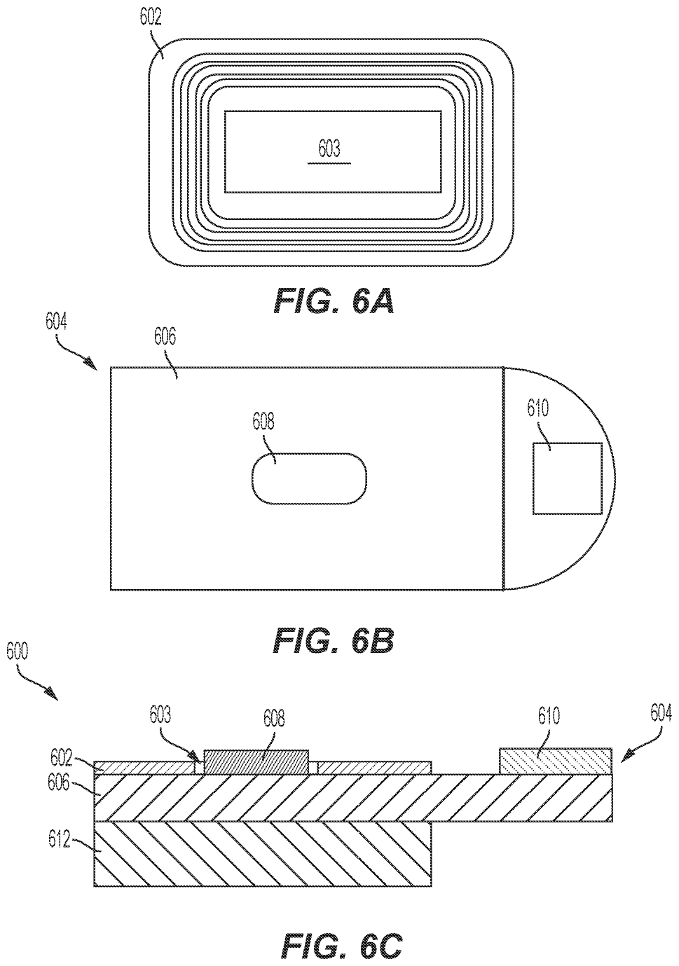

FIGS. 6A-6C are schematic illustrations of certain components of an implantable device 600 in accordance with the present technology. Components of the implantable device 600 that are not shown in these figures include a housing, reservoir, septum, an outlet port, and other components disclosed herein. Any of the implantable devices disclosed herein can include the components depicted in FIGS. 6A-6C. Likewise, the implantable device 600 can include any of the components or features of other implantable devices disclosed herein.

As shown in FIG. 6A, the device 600 can comprise an antenna 602. The antenna 602 can comprise a coil, which can take the form of a loop of conductive wire or other material that is embedded within an insulative material. In some embodiments, the coil can define an aperture 603 of the antenna 602. The aperture 603 can be configured to receive one or more other electronic components at least partially therein. Although the antenna 602 shown in FIG. 6A has a rounded rectangular shape, other shapes are possible. For example, the antenna 602 can be substantially circular, ovular, triangular, polygonal, etc. According to various embodiments, the antenna 602 can be configured to receive and/or emit electrical energy. For example, the antenna 602 can comprise an NFC antenna 602 configured for near field communication. In some embodiments, the antenna 602 is configured to inductively couple with an interrogation device (or a component thereof). The antenna 602 can be electrically coupled to a controller (e.g., a microcontroller, a chip, etc.).

The implantable device 600 can further include a circuit board assembly 604, for example as shown in the top view depicted in FIG. 6B. The circuit board assembly 604 can include a printed circuit board 606 or other suitable substrate for carrying (e.g., disposed on, adhered, secured, etc.) one or more electronic components. The printed circuit board 606 can carry a sensing element 608 and/or a wireless transceiver 610. In some embodiments, for example as shown in FIG. 6B, the sensing element 608 and/or the wireless transceiver 610 can be disposed on a top surface of the printed circuit board 606. As but one example, the sensing element 608 can comprise an optical pulse oximeter and/or the wireless transceiver 610 can comprise a Bluetooth Low Energy transceiver. Still, in other embodiments different sensing elements and/or wireless transceivers can be used. Although FIG. 6B depicts one sensing element 608 and one wireless transceiver 610, there may be additional sensing elements 608 and/or wireless transceivers 610 carried by the device 600. In some embodiments, a battery 612 is carried by the printed circuit board 606 and configured to supply power to one or more of the various electronic components of the implantable device 600.

Although FIG. 6B depicts the printed circuit board 606 having one portion with a rectangular shape and another portion with a semicircular shape, many other shapes of the printed circuit board 606 are possible. In some embodiments, a shape of the printed circuit board 606 and/or one or more portions thereof can be based, at least in part, on a shape of a housing of the implantable device 600 that is configured to carry the printed circuit board 606. For example, the printed circuit board 606 can be shaped and/or sized to fit within a cavity defined between an outer surface of the implantable device 600 and a sidewall of a reservoir of the implantable device 600.

The electronic components disclosed herein (and others) can be arranged in various configurations. FIG. 6C schematically illustrates a side cross-sectional view of an example configuration. As shown in FIG. 6C, the antenna 602 can be disposed on the printed circuit board 606, which can be disposed on the battery 612 (e.g., the battery 612 is positioned at a bottom side of the printed circuit board 606). In FIG. 6C, the antenna 602 includes the aperture 603 such that one or more of the sensing elements 608 (or transceivers, other components, etc.) can be positioned at least partially within the aperture 603. Such configuration may facilitate or permit the antenna 602 to be positioned substantially flush with a top surface of the printed circuit board 606.

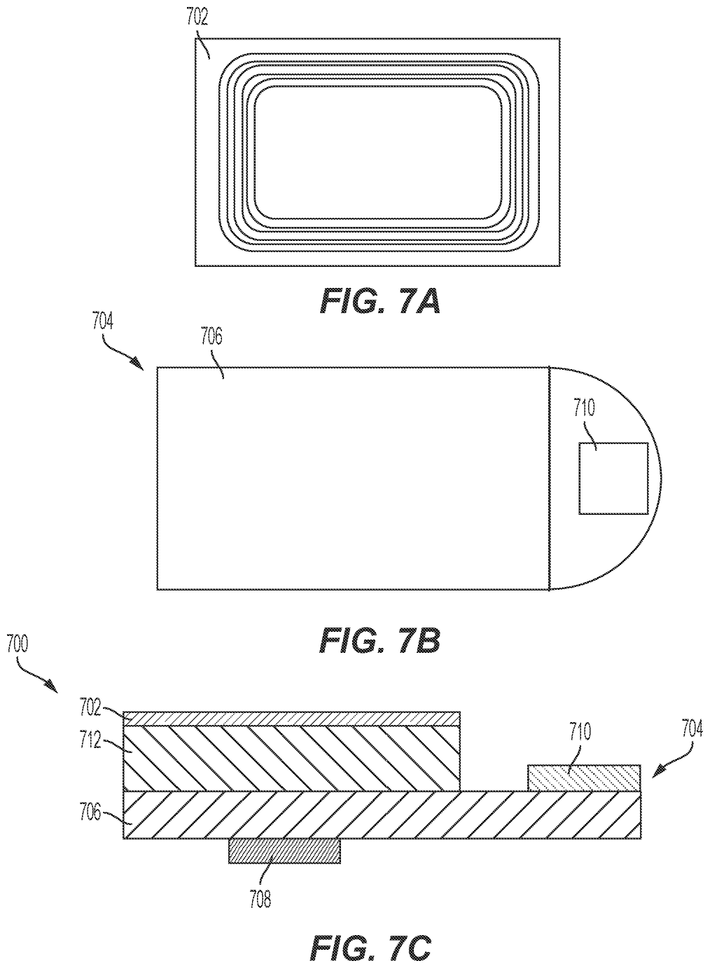

FIGS. 7A-7C are schematic illustrations of certain components of an implantable device 700 in accordance with the present technology. Components of the implantable device 700 that are not shown in these figures include a housing, reservoir, septum, an outlet port, and other components disclosed herein. In some embodiments, any of the implantable devices disclosed herein can include the components depicted in FIGS. 7A-7C. Likewise, the implantable device 700 can include any of the components or features of other implantable devices disclosed herein.

An antenna 702 of the present technology can comprise a coil, which can take the form of a loop of conductive wire or other material that is embedded within an insulative material. In some embodiments, for example as shown in FIG. 7A, the antenna 702 does not comprise an aperture. Although the antenna 702 shown in FIG. 7A has a rectangular shape, other shapes are possible. The antenna 702 can be configured to receive and/or emit electrical energy. For example, the antenna 702 can comprise an NFC antenna 702 configured for near field communication. In some embodiments, the antenna 702 is configured to inductively couple with an interrogation device (or a component thereof). The antenna 702 can be electrically coupled to a controller (e.g., a microcontroller, a chip, etc.).

The implantable device 700 can further include a circuit board assembly 704, for example as shown in the top view depicted in FIG. 7B. The circuit board assembly 704 can include a printed circuit board 706 or other suitable substrate for carrying one or more electronic components. The printed circuit board 706 can carry a sensing element 708 (positioned at a bottom side of the printed circuit board 706 and not visible in FIG. 7B) and/or a wireless transceiver 710. The wireless transceiver 710 can be disposed on a top surface of the printed circuit board 706 (as shown in FIG. 7B) or disposed on a bottom surface of the printed circuit board 706. In some embodiments, a battery 712 is carried by the printed circuit board 706 and configured to supply power to one or more of the various electronic components of the implantable device 700.

The electronic components disclosed herein (and others) can be arranged in various configurations. FIG. 7C schematically illustrates a side cross-sectional view of an example configuration. As shown in FIG. 7C, in some embodiments the antenna 702 can be disposed on the battery 712, which can be disposed on the top surface of the printed circuit board 706.

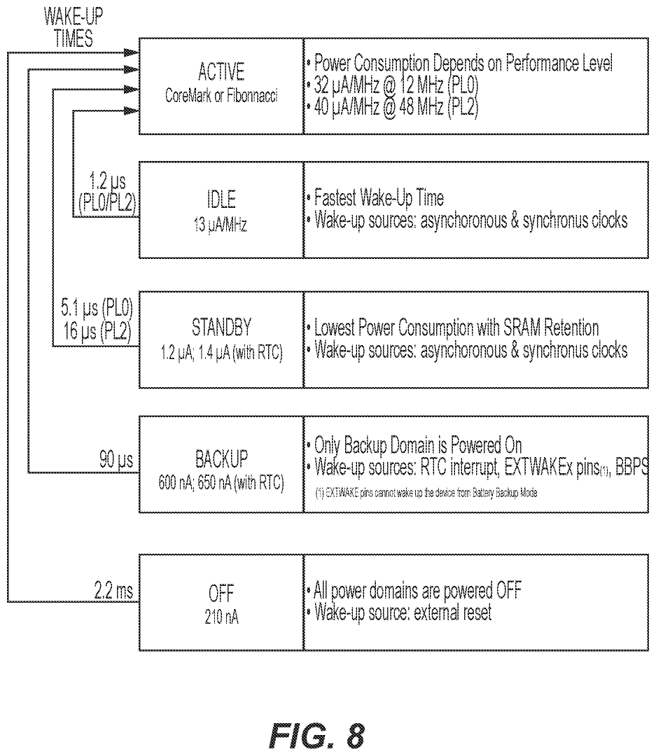

In various embodiments, an implantable device of the present technology can modulate its operation (e.g., to increase or decrease power consumption) based on certain input conditions or trigger events. FIG. 8 illustrates five exemplary power states that can be assumed by an implantable device and/or a component thereof (e.g., any of the controllers disclosed herein). Such power states can include an active state, an idle state, a standby state, a backup state, an off state, etc. The five exemplary power states shown in FIG. 8 range from the most power-intensive (e.g., the “Active” state in which data is both collected and transmitted to an external device) to the least power-intensive (e.g., the “off” state in which little to no battery power is used). As illustrated, the time required for the implantable device to “wake up” from the various low-powered states to the fully operational “active” state increases with more low-power states (e.g., the time required to wake up from the “Off” state is greater than the time required to wake up from the “Backup” state). The various states and power consumption values depicted here are exemplary only, and serve to illustrate the various types of low-power states that the implantable device may obtain, as well as the various techniques that can be used to wake up the implantable device from these low-power states to a fully operational “Active” state.

As shown in FIG. 8, in the “Active” state the power consumed by the device or controller is a function of the frequency of operation. For example, at 12 MHz the power consumption is 32 μA/MHz, but at 48 MHz the power consumption is 40 μA/MHz. Thus, power consumption can be modulated by modifying clock frequency. However, reducing the clock frequency can also reduce performance. In the “Active,” “Idle,” and “Standby” states, sufficient power can be supplied to volatile memory such as SRAM to support storage of data in the volatile memory. In the “Idle” state, the power consumption can be approximately 13 μA/MHz, which is less than the power consumption in the “Active” state at either 12 MHz or 48 MHz. In the “Standby” and “Backup” states, the power consumption can vary based on whether a real time clock (RTC) is used. For example, the power consumption in the “Standby” state with RTC can be approximately 1.4 μA while the power consumption in the “Standby” state without RTC can be approximately 1.2 μA and/or the power consumption in the “Backup” state with RTC can be approximately 650 nA while the power consumption in the “Backup” state without RTC can be approximately 600 nA. In the “Off” state, the power consumption can be about 210 nA.

Increasing the power state from “Idle” to “Active” can take approximately 1.2 μS, which is the fastest of the depicted wake-up times. For example, increasing the power state from “Standby” to “Active” can take between approximately 5.1 μS to 16 μS based on the clock frequency, increasing the power state from “Backup” to “Active” can take approximately 90 μS, and/or increasing the power state from “Off” to “Active” can take approximately 2.2 mS.

In the “Idle” state and/or the “Standby” state, a change in power state can be caused by asynchronous and/or synchronous clocks. In the “Backup” state, a change in power state can be caused an interruption of an RTC that can measure time even when a controller is powered off, an interruption of a wake up interrupt controller (e.g., EXTWAKEx pins as shown in FIG. 8), and/or instructing a battery backup power switch (BBPS). In the “Off” state, a change in power state may be caused by an external reset of the device and/or controller.

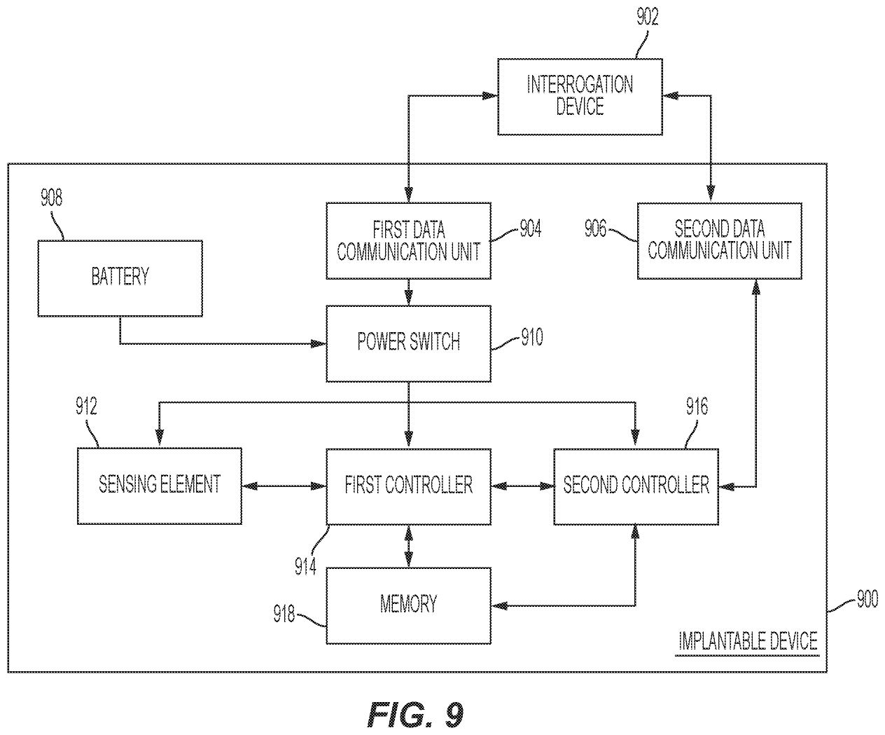

FIG. 9 is a schematic block diagram of an environment for communication between an implantable vascular access device 900 (“device 900”) and an interrogation device 902. The implantable device 900, the interrogation device 902, and/or components of the implantable device 900 and/or the interrogation device 902 can be similar to corresponding devices and components disclosed herein, except as detailed below. As shown in FIG. 9, device 900 can comprise a first data communication unit 904 and a second data communication unit 906, each of which can be configured to communicate with the interrogation device 902. Although FIG. 9 depicts two data communication units, the device 900 can include any suitable number of data communication units (e.g., one, three, four, etc.). The device 900 can comprise a battery 908 configured to provide electrical power to one or more components of the device 900. In some embodiments, the battery 908 is electrically coupled to one or more components of the device 900 via one or more power switches 910 configured to control the communication of electrical energy between the battery 908 and the one or more components of the device 900. As shown in FIG. 9, such one or more components can include one or more sensing elements 912, a first controller 914, a second controller 916, and/or other suitable components. Although FIG. 9 depicts two controllers, the device 900 can include any suitable number of controllers (e.g., one, three, four, etc.).

In some embodiments, one or more of the sensing elements 912 are electrically coupled to the battery 908 via a first one of the power switches 910, the first controller 914 is electrically coupled to the battery 908 via a second one of the power switches 910, and the second controller 916 is electrically coupled to the battery 908 via a third one of the power switches 910. In various embodiments, one of the power switches 910 can electrically couple the battery 908 to two or more of the sensing elements 912, the first controller 914, and/or the second controller 916. The device 900 can include memory 918 that is electrically coupled to the first controller 914 (see FIG. 9), the second controller 916 (see FIG. 9), the sensing elements 912 (not shown in FIG. 9), and/or other components of the device 900. The memory 918 can include read-only memory (ROM) and/or random access memory (RAM) and/or other storage devices such as SSDs that store executable applications, software, instructions, data, databases and/or other software required to, for example, implement the various routines described herein, control device components, store data, and/or communicate and exchange data and information with remote computers and other devices.

In some embodiments, communication between the interrogation device 902 and the first data communication unit 904 can consume less power than communication between the interrogation device 902 and the second data communication unit 906. For example, communication via a Bluetooth communication standard or similar may consume significantly more power than communication via NFC or a similar standard. However, transmission of data via a communication link requiring higher power (e.g., Bluetooth, etc.) may be desirable for speed, data quality, transmission range, etc. Thus, the interrogation device 902 and the first data communication unit 904 may be configured to communicate with one another according to a first wireless communication link that requires lower power for activation and/or modulation of operation of the device and the interrogation device 902 and the second data communication unit 906 may be configured to communicate with one another according to a second wireless communication link requiring more power. Additionally or alternatively, the first controller can be configured to consume less power than the second controller. According to various embodiments, the first data communication unit 904 can be configured to communicate via NFC communication standard and/or the second data communication unit 906 can be configured to communicate via Bluetooth standard. In some embodiments, the first data communication unit 904 is configured to receive a modulation signal (e.g., from an interrogation device 902, from a component of the implantable device 900, from other implantable or wearable devices, etc.). The second data communication unit 906 can be configured to receive data from one or more electronic components of the implantable device 900 (e.g., the second controller 916, the memory 918, the first controller 914, etc.) and transmit the data to another device (e.g., interrogation device 902, remote computing devices, other implantable or wearable devices, etc.).

In some embodiments, power can be supplied from a battery 908 of the implantable device 900 to the sensing elements 912, the first controller 914, the second controller 916, and/or other electrical components of the implantable device 900. In some embodiments, the battery 908 can be indirectly coupled to one or more electrical components via one or more power switches 910 (see FIG. 9). For example, the power switch 910 can allow, facilitate, and/or increase the flow of electrical energy from the battery 908 to at least one of the one or more sensing elements 912, the first controller 914, or the second controller 916 after the first data communication unit 901 has received a modulation signal. Each of the one or more sensing elements 912, the first controller 914, or the second controller 916 can be indirectly coupled to the battery 908 via a distinct power switch 910 and/or one or more of the one or more sensing elements 912, the first controller 914, or the second controller 916 can be indirectly coupled to the battery 908 via the same power switch 910. In some embodiments, at least one of the one or more sensing elements 912, the first controller 914, or the second controller 916 can be directly electrically coupled to the battery 908.

As noted with reference to FIG. 7, in some embodiments the device 900 and/or one or more components thereof can have a low power state in which certain functionalities of the device 900 and/or component(s) are limited and/or modulated. For example, in some embodiments the second controller 916 can remain in a low power state until a modulation signal is received by the first data communication unit 904. In various embodiments, while in the low power state at least some components or functions of the device 900 may be inoperative. Once the modulation signal has been received, the power switch 910 electrically coupled to the second controller 916 can permit greater power to be transmitted from the battery 908 to the second controller 916 causing the second controller 916 to enter a higher power state and enabling the second data communication unit 906 to transmit data from the second controller 916. In some embodiments, the second controller 916 only maintains the higher power state for a limited duration of time. According to various embodiments, one or more sensing elements 912 can be configured to obtain data continuously and/or periodically. In some embodiments, data collection by the sensing elements 912 is at least partially independent of any modulation signal (or lack thereof) received by the first data communication unit 904. Additionally or alternatively, receipt of a modulation signal by the first data communication unit 904 can allow, facilitate, and/or modulate data collection by the sensing elements 912. For example, in some embodiments a sensing element 912 can obtain data at a baseline frequency. If a modulation signal is received by the first data communication unit 904, the first controller 914 can cause the sensing element 912 to obtain data at a greater frequency to capture more and/or higher fidelity data. In some examples, a sensing element 912 may not collect data in the absence of a modulation signal. Rather, once a modulation signal is received by the first data communication unit 904, the first controller 914 can cause the sensing element 912 to temporarily obtain data (e.g., for a predetermined amount of time, for a predetermined number of samples, until the modulation signal is no longer detected by the first data communication unit 904, etc.).

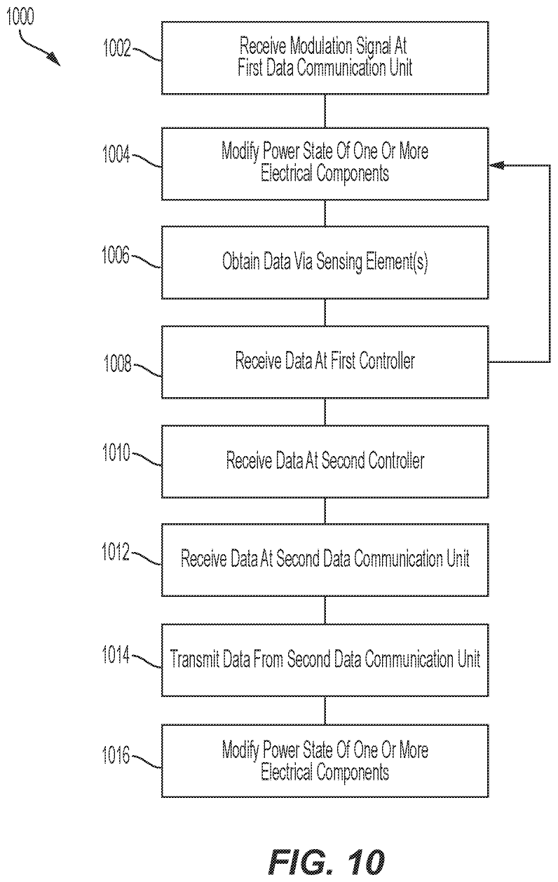

FIG. 10 is a flow diagram of an example process 1000 for obtaining and transmitting data via an implantable device, such as the implantable device 900 described with reference to FIG. 9 and/or others. The process 1000 can comprise receiving a modulation signal at a first data communication unit (process portion 1002). In some embodiments, the modulation signal is emitted by an interrogation device. For example, the modulation signal can comprise electrical energy emitted by an antenna of an interrogation device that is, optionally, positioned in close proximity to the implantable device. Still other modulation signals are possible, as disclosed herein.

At process portion 1004, the process 1000 can include modifying a power state of one or more electrical components of the implantable device after receiving the modulation signal and/or in response to the modulation signal. Such electronic components can comprise one or more sensing elements and/or one or more controllers (e.g., the first and second controllers 914, 916, the controller 506, etc.). Modifying a power state of an electrical component can comprise regulating the flow of power from a battery to the component. For example, in response to the modulation signal, the process 1000 can include changing a configuration of a power switch that is electrically coupled to a battery and an electrical component such that more electrical energy is allowed to flow from the battery to the component. In some embodiments, a power state of an electrical component can be modified based on data received by one or more controllers of the implantable device. In various embodiments, the first controller can cause an increase in power state of the second controller based on physiological data received by the first controller. For example, if the first controller receives physiological data from a sensing element and determines that a patient's oxygen level is abnormal, the first controller can cause a change in power state of the second controller such that the second controller transmits the physiological data to a remote computing device where the data can be interpreted and/or monitored by a user (e.g., the patient, a clinician, etc.). Thus, these and other changes in power state may not be caused by receipt of a modulation signal from an external interrogation device.

Referring still to FIG. 10, at process portion 1006 the process 1000 can include obtaining data via one or more sensing elements of an implantable device. In some embodiments, one or more sensing elements can be configured to obtain data continuously and/or periodically. In some embodiments, data collection by the sensing elements is at least partially independent of any modulation signal (or lack thereof) received by the first data communication unit. Additionally or alternatively, receipt of a modulation signal by the first data communication unit can allow, facilitate, and/or modulate data collection by the sensing elements.

At process portion 1008, the process 1000 can include receiving data at a first controller. In some embodiments, one or more sensing elements of the implantable device are configured to communicate data obtained by the one or more sensing elements to the first controller. The first controller can be an ultra-low power controller as disclosed herein, or any other suitable controller. In some embodiments, the first controller and/or the one or more sensing elements can be configured to communicate the data to a separate memory of the implantable device. Additionally or alternatively, the data can be stored in a memory of the first controller and/or the one or more sensing elements.

In some embodiments, an implantable device of the present technology can comprise a first controller configured to consume a small amount of power and a second controller configured to consume a larger amount of power. For example, as described with reference to FIG. 9, a device can comprise a first controller configured to communicate with sensing elements of the device using less power for a given frequency of operation and a second controller configured to communicate with a second data communication unit for transmitting data to an interrogation device and/or a remote computing device, which may require greater power at a given frequency of operation. Thus, at process portion 1010 the process 1000 can include receiving data at a second controller and at process portion 1012 the process 1000 can include receiving data at a second data communications unit. In some embodiments, the first controller is configured to transmit the data to the second controller. Additionally or alternatively, the data can be communicated from a separate memory of the implantable device to the second controller. In various embodiments, one or more sensing elements can be configured to transmit data to the second controller.

At process portion 1014, the process 1000 can include transmitting data from the second data communication unit. For example, the second data communication unit can transmit data to an interrogation device and/or a remote computing device. In some embodiments, for example as shown in FIG. 10, at process portion 1016 the process 1000 can include modifying a power state of one or more electrical components. For example, after transmitting data from the second data communication unit (e.g., to a remote computing device, etc.) the process 1000 can include causing one or both of the first and second controllers to enter a power state in which the first and/or second controller consumes less power.

A modulation signal of the present technology can comprise many forms. For example, the modulation signal comprises electrical energy emitted by the interrogation device and received by the first data communication unit. In some embodiments, the implantable device can comprise a sensing element configured to detect a modulation signal. For example, the implantable device can comprise a capacitive switch, a Hall-Effect sensor, an inductive sensor, or another non-contact sensor. Additionally or alternatively, the modulation signal can comprise a force, a moment, a pressure, etc. applied to the implantable device and detected by a sensing element carried by the implantable device. For example, the implantable device can comprise a transducer configured to detect pressure applied to implantable device through the patient's skin (e.g., via a user physically tapping the device, etc.). In some embodiments, the implantable device can comprise a button configured to be pressed by a user through the patient's skin such that, when the button is pressed, a modulation signal is generated and detected.

In any of the embodiments disclosed herein, operation of one or more of the electronic components of the implantable device (e.g., the sensing elements, either of the first and second controllers, etc.) can be configured to be modulated (e.g., to exit low power mode, to collect data, etc.) by a modulation signal having one or more specific parameters. For example, in embodiments in which the modulation signal comprises a pressure applied to the implantable device by a user pressing on the device through the patient's skin, operation of the one or more electronic components can be modulated if the modulation signal comprises a predetermined number of period of increased pressure (e.g., “taps”) on the device, a predetermined number of period of increased pressure (e.g., “taps”) on the device in a specific sequence and/or at a specific frequency, pressure applied to the device for at least a predetermined period of time, etc.

In some embodiments, the modulation signal can comprise a physiological parameter, a change in a physiological parameter, a pattern of a physiological parameter, etc. The physiological parameter can be detected by a sensing element carried by the implantable device and/or a sensing element carried by another device such as a wearable device (e.g., a smartwatch, etc.) and/or another implantable device (e.g., a pacemaker, an insulin pump, etc.). In some embodiments, a device of the present technology (e.g., device 100) can communicate with other implantable and wearable devices. For example, a wearable device can obtain physiological data as a gross measurement tool that can be continuously screening for abnormal data and recharged often. Such wearable device could, in turn, send a modulation signal to the implantable device to cause the implantable device to obtain and/or transmit high fidelity data. The modulation signal can comprise a motion parameter and/or an activity parameter, a heart rate parameter, a respiration rate parameter, a temperature parameter, a blood oxygen saturation parameter, etc. Activation of one or more electronic components of the implantable device in response to a physiological parameter or derivative thereof can be useful for predicting and/or treating a medical condition. For example, the implantable device can be activated to collect and/or transmit data in response to an increase in heart rate and temperature that may indicate that the patient is unwell. Such data can be communicated to the patient and/or a caregiver (e.g., via the higher power communication link).

According to various embodiments, a physiological parameter can be intentionally modified by a user to activate the device. For example, the modulation signal can comprise a change in temperature of at least a predetermined magnitude that is detected by a sensing element of the implantable device. In these and other embodiments, a user can place an ice pack or a heating pad on the patient's skin over the implantable device to cause a change in temperature sufficient to activate the device. In some examples, the modulation signal can comprise a specific movement of the patient. For example, a patient's smartwatch can determine that a patient is performing a specific movement (e.g., waving of the hand in a specific direction, turning of the wrist a specific number of times, etc.) and can communicate a modulation signal to the implantable device in response to the movement.

In some embodiments, an implantable device in accordance with the present technology can be configured to modulate its operation in response to audio data. For example, an implantable device can comprise one or more microphones or any other transducer configured to convert sound waves into an electrical signal. The one or more microphones can be electrically coupled to any of the controllers disclosed herein and/or include a controller configured to process the audio data. As but one example, a user or a device (e.g., an interrogation device, a remote computing device, etc.) can generate a voice command that is detected by the microphone. A controller can determine if the voice command is known and/or trusted and based on the determination, cause the device or a component thereof to modulate its operation in accordance with predefined logic. For example, a patient can say “Device, send data to my phone” aloud, this sound wave can be detected and converted into audio data by a microphone carried by an implantable device, a controller can increase a power state of a controller and/or data communications unit configured to transmit data to an interrogation or remote device using substantial power. Additionally or alternatively, a patient can say “Device, enter low power mode” aloud and one or more electrical components of an implantable device can enter a low power mode and/or the flow of electrical energy to the one or more components can be limited (e.g., via a power switch). In some embodiments, the controller can determine if the voice command is known and/or trusted based on a characteristic of the audio data (e.g., a wavelength, an amplitude, a pitch, etc.).

Patient healthcare often involves multiple stakeholders including the patient themselves, loved ones of the patient, caregivers of the patient, medical professionals, etc. Thus, it may be useful for a stakeholder in the patient's care to be able to obtain data regarding a health of the patient, a health of the patient's implantable device, etc. In some embodiments, a modulation signal of the present technology can comprise a request from a stakeholder in the patient's care. For example, a clinician can send a request for data from a remote computing device (e.g., a mobile phone, a computer, etc.) to the implantable device. Such request for data can be sent to the implantable device via a wireless communication link such as, but not limited to, near-field communication (NFC), infrared wireless, Bluetooth, ZigBee, Wi-Fi, inductive coupling, or capacitive coupling. In some embodiments, the communication link can be secured and/or trusted to protect sensitive patient data. Additionally or alternatively, the implantable device and/or a remote commuting device can be configured to generate a notification or alert to a patient, caregiver, clinician, etc. when a request for data has been sent to the implantable device. Such notifications may comprise audible, visual, and/or tactile notifications.

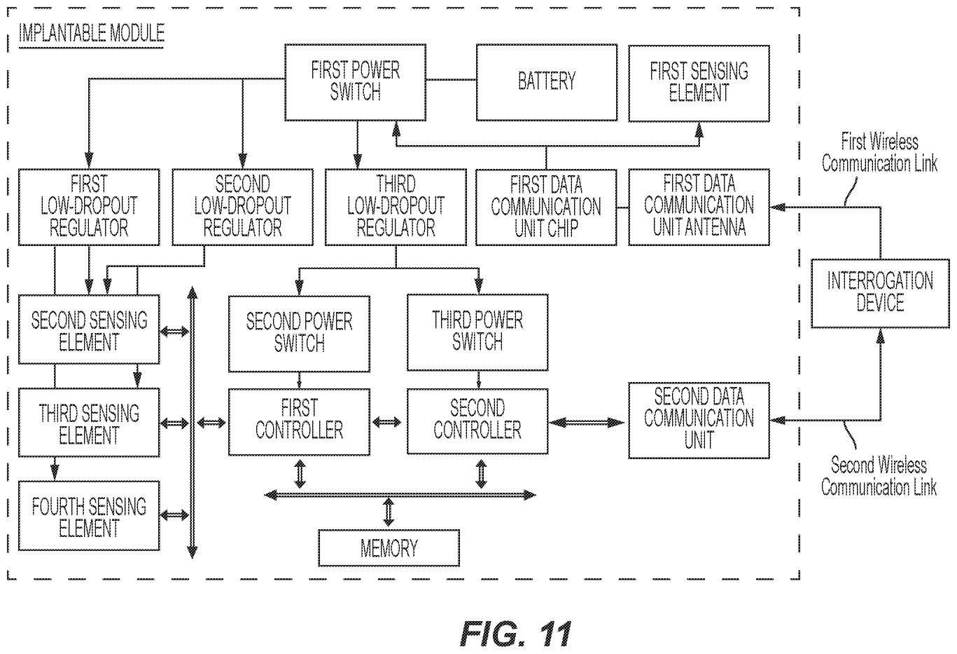

FIG. 11 is a schematic diagram of an implantable device in communication with an interrogation device. As shown, the implantable module (e.g., an implantable vascular access device) can be configured to communicate with the nearby interrogation device (e.g., a smartphone or other mobile device) via a first wireless communication link and a second wireless communication link. In some embodiments, the first and second wireless communication links comprise two different wireless communication links. In some embodiments, communication via the first wireless communication link consumes less power than communication via the second wireless communication link. In these and other embodiments, the first wireless communication link can comprise an NFC link and the second wireless communication link can comprise a Bluetooth communication link (e.g., Bluetooth Low Energy, etc.). As shown in FIG. 11, the interrogation device can be configured to communicate with an antenna of a first data communication unit of the implantable module via the first wireless communication link. For example, in embodiments in which the first wireless communication link comprises an NFC link, the first data communication antenna can comprise an NFC antenna. The first data communication unit antenna can be electrically coupled to a first data communication unit chip (for example as shown in FIG. 11) and/or the first data communication chip can comprise an integrated first data communication antenna. In some embodiments, the first data communication unit chip can be electrically coupled to a sensing element (e.g., the First Sensing Element in FIG. 11). Such sensing element can be at least partially powered by electrical energy received by the first data communication unit antenna and thus, may be externally powered without the need for battery-supplied power.

The first data communication chip can be electrically coupled to a power switch that is electrically coupled to a battery and/or one or more additional electronic components. As shown in FIG. 11, the power switch can be electrically coupled to one or more additional power switches, one or more low-dropout regulators, one or more sensing elements, one or more controllers, or other electronic components. Thus, such electronic components can be configured to receive electrical energy from the battery via the power switch and/or the power switch can at least partially control or regulate delivery of electrical energy from the battery to the electrical components. As shown in FIG. 11, the power switch can be electrically coupled to low-dropout regulators. One, some, or all of the low-dropout regulators can be electrically coupled to one or more sensing elements and/or one or more additional power switches. In some embodiments, a low-drop regulator can be electrically coupled to a first power switch, which is in turn electrically coupled to a first controller, and/or a second power switch, which is in turn electrically coupled to a second controller. In any of the embodiments disclosed herein, an implantable device can include a memory, which can be electrically coupled to one or more controllers (see FIG. 11, for example).

As shown in FIG. 11, in some embodiments one or more of the sensing elements are electrically coupled to the first controller, which can in turn be electrically coupled to the memory and/or the second controller. The second controller can be electrically coupled to the first controller, the memory, and/or the second data communication unit. The second data communication unit can be configured to communicate with another device (e.g., an interrogation device, a remote computing device, etc.) via the second wireless communication link. In some embodiments, the second data communication unit comprises a chip and/or an antenna. Thus, electrical energy and thereby data can be transmitted from the sensing element(s) to the first controller to the memory and/or to the second controller, from the second controller to the other device via the second data communication unit.

In operation, the interrogation device can communicate with the implantable module via the first wireless communication link. The first data communication unit chip, coupled to the first data communication unit antenna, can initiate a power switch reset, which may optionally cause the sensing elements to obtain data and/or modulate how the sensing elements obtain data. The data obtained by the sensing elements can be transmitted to the first controller and, optionally, from the first controller to the memory. The second controller can receive data from the first controller and/or the memory, and can transmit the data stored to the interrogation device via the second wireless communication link, for example by transmission via the second data communication unit. In this manner, the implantable module may operate in a low-power state (e.g., in which data is collected via sensing elements but is not transmitted wirelessly, in which data is not actively collected by the sensing elements, etc.) until the modulation signal is received via the first wireless communication link, at which point the implantable module can transmit data via the second wireless communication link.

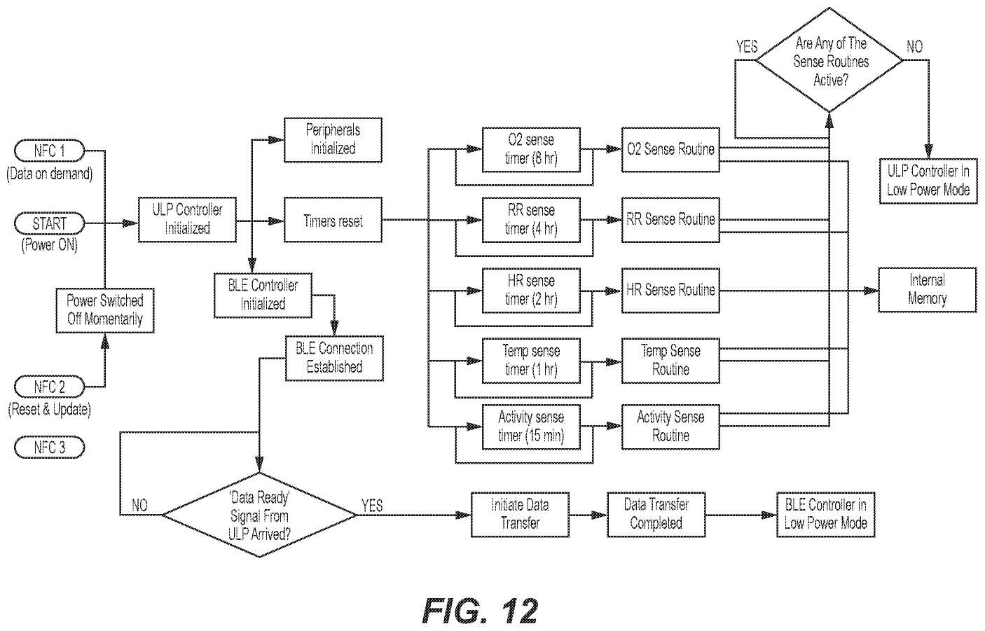

FIG. 12 is an example flow diagram illustrating operation of an implantable device in accordance with the present technology. Although specific communication modalities (e.g., NFC, BLE) and sensing modalities (e.g., 02, RR, HR, temp, activity, etc.) are shown in FIG. 12, other communication and/or sensing modalities are possible. For example, another electromagnetic coupling and/or low-power communication standard can be used instead of and/or in addition to NFC. In some embodiments, another direct radio transmission and/or higher-power communication standard can be used instead of and/or in addition to Bluetooth Low Energy. Similarly, although specific numbers of components (e.g., NFC 1, NFC 2, NFC 3, NFC 4, etc.) and types of components (e.g., ULP controller, BLE controller, etc.) are depicted, other numbers and types of such components are possible. Numerical parameters shown in FIG. 12 (e.g., 8 hours, 15 minutes, etc.) are intended to be exemplary. Such numerical parameters are non-exhaustive and may be substituted with other appropriate numerical parameters.

As illustrated, the flow can begin with receipt of an NFC signal (“NFC 1”) or initial powering on of the device, which causes the ultra-low power microcontroller (ULP controller) to be initialized. This initialization can also take place if an NFC signal (“NFC 2”) is received that causes a reset and update, in which case the power is switched off momentarily. After initialization, peripherals of the implantable device are initialized, timers are reset, the Bluetooth Low Energy controller is initialized and the connection with an interrogation device is established. If the controller indicates that data is ready to be transmitted, then data transfer is initiated (e.g., via a Bluetooth Low Energy transceiver). After the data is transmitted, the Bluetooth Low Energy controller can be returned to a low-power mode until it is initialized again.

Returning to the initialization, after the timers are reset, various sensors can collect data according to various timing configurations (e.g., oxygen levels measured every 8 hours, heart rate detected every 2 hours, etc.). These measurements can be stored in internal memory for transmission via the Bluetooth link. If any of the sensing routines are active, the controller stays in its active state. But when none of the routines are active, the controller can revert to a low-power mode so as to conserve energy.

The flow illustrated in FIG. 12 is but one example of maintaining certain components of an implantable device in low-power states for some time interval, and selectively transitioning those components to fully operational states only at certain predetermined times so as to conserve power while also transmitting useful data to a nearby interrogation device. In various embodiments, the particular components can vary, as well as the triggers or other conditions that cause the components to transition between high-power and low-power states.

Selected Examples of Mobile Device Applications for Use with Implantable Devices

As noted previously with respect to FIG. 1, a system of the present technology (e.g., system 10) may include first remote computing device(s) 160 which can be in communication with the local computing device 150, which in turn may be in communication with the implantable device 100. In some embodiments, the first remote computing device(s) 160 can include, for example, server computers associated with a hospital, a medical provider, medical records database, insurance company, or other entity charged with securely storing patient data and/or device data. At the remote location 170 (e.g., a hospital, clinic, insurance office, medical records database, operator's home, etc.), an operator may access the data via a second remote computing device 172, which can be, for example a personal computer, smart device (e.g., a smartphone, a tablet, or other handheld device having a processor and memory), or other suitable device. The operator may access the data, for example, via a web-based application. In some embodiments, the obfuscated data provided by the device 100 can be de-obfuscated (e.g., unencrypted) at the remote location 170.

As noted above, an implantable device 100 may communicate with a mobile application (e.g., software running on the local computing device 150 such as a smartphone or table) that will provide the patient with a user interface serving multiple functions. On the application, patients can view and track their own data and metrics coming from the implantable device 100. Patients can also enter information regarding their symptoms as it pertains to their cancer and oncologic therapy. From the application, patients can directly contact their care team or any family members and other designated caregivers in the event of an emergency. Finally, patients will be able to learn about their symptoms and how to manage them at home through educational resources on the application.

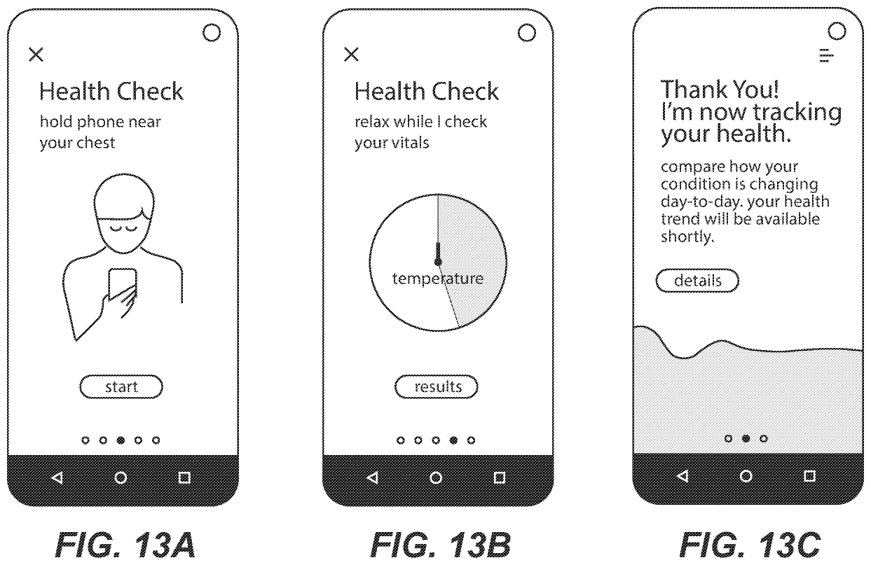

FIGS. 13A-13C illustrate example user interfaces for a user to perform a health check using the mobile device as an interrogation device. As shown in FIG. 13A, the application can prompt the user to hold the phone near the user's chest (or adjacent wherever the implantable device is located). FIG. 13B illustrates a graphic that can be displayed while the interrogation device interacts with the implantable device (e.g., transmitting a wake-up signal via NFC), and while the implantable device collects and transmits data to the interrogation device (e.g., via a Bluetooth link). When the user selects the “results” button, the interface can transition to that shown in FIG. 13C, which allows a user to see details. The interface can also include a graphical representation of a user's measurements over time, as shown in the sloping line along the lower portion of the screen. This graphical representation can provide a quickly discernible signal to the patient that can be particularly useful if a patient's parameters (e.g., body temperature, oxygen levels) are dropping or falling significantly relative to the patient's historical baseline. In some embodiments, after the patient's physiological data has been transmitted to the interrogation device and displayed to the user, the implantable device can revert to the low-power or standby state.

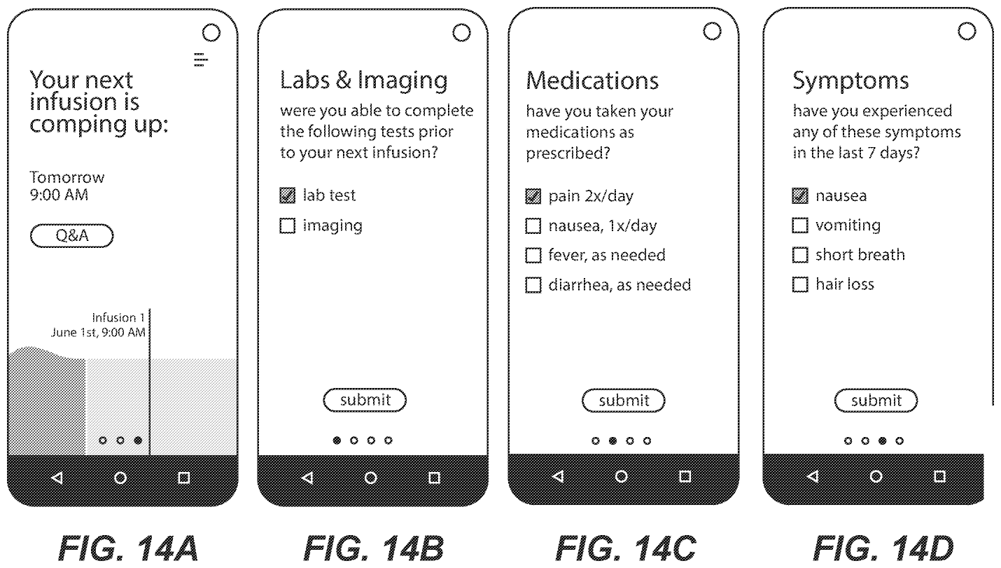

FIGS. 14A-14C illustrate additional user interfaces for a software application, here related to various outputs and inputs for a patient. In FIG. 14A, a reminder is presented for a patient's upcoming appointment. Once the user selects “Q&A” on the interface, the user can be prompted to provide certain information (e.g., via the interfaces shown in FIGS. 14B-14D) such as whether the user has completed the requested lab tests or imaging appointments, taken prescribed medications, or experienced various symptoms. In some embodiments, a symptoms interface can also allow a user to provide other inputs such as an image (e.g., captured via a camera of the mobile device), an audio file (e.g., a recording of the user's voice describing their state or symptoms), or any other suitable user input. By collecting such information remotely, a clinician can monitor a patient's progress without requiring the patient to travel to the hospital or clinic.

In various embodiments, remote monitoring systems as disclosed herein can analyze data collected via implantable devices 100. In some embodiments, a plurality of different implantable devices 100 can be accessed to aggregate large amounts of data on cancer patients and their outcomes. Machine learning and deep learning technology may then be employed to identify the matters associated with the onset of complications such as sepsis. Next, artificial-intelligence-enabled algorithms may be utilized to prospectively identify patients showing early signs of complications and clinical decline, alerting the care team and triggering timely interventions when the benefits are maximized. Such data analytics solutions may be particularly valuable for third parties such as health systems and pharmaceutical companies as they will better understand the outcomes of their patients and can use data to improve care and reduce costs.

In some embodiments, a remote monitoring system as described herein can be integrated into the current oncology workflow, placing the collected and analyzed data at the hands of the clinical team. In some embodiments, the data generated from the implantable device and/or the system can be pushed directly to the hospital/medical system electronic medical record where it can be viewed in the patient chart in the same location as the rest of the patient data. Vital sign data can populate preexisting flowcharts showing the clinical team what is happening to the patients between visits. Alerts coming from the system may be generated when a measurement from the device falls outside a specified range (i.e. temperature above a certain threshold indicating a fever). These alerts may go directly to the clinic's existing alert system for triage during working hours. After hours, alerts can be sent to either the on-call physician or to a telemedicine service such as Doctor on Demand.

### CONCLUSION

Although many of the embodiments are described above with respect to vascular access devices for patient monitoring, the technology is applicable to other applications and/or other approaches, such as other types of implantable medical devices (e.g., pacemakers, implantable cardioverter/defibrillators (ICD), deep brain stimulators, insulin pumps, infusion ports, orthopedic devices, and monitoring devices such as pulmonary artery pressure monitors). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above.

The above detailed descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

For the purposes of this specification and appended claims, unless otherwise indicated, all numbers expressing temperatures, percentage changes in physiological parameters, concentration of blood constituents, heart rate, respiratory rate, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An implantable vascular access device comprising:
a fluid reservoir;
a cover disposed over the reservoir;
an outlet port configured to mate with a catheter, the outlet port fluidically coupled to the fluid reservoir;
one or more sensors configured to capture physiological data while the device is implanted within a patient;
a first wireless transceiver configured to transmit the physiological data to one or more external devices via a first communication link; and
a second wireless transceiver configured to communicate with one or more external devices via a second communication link;
wherein the device is configured to transition between a low-power first state and a higher-power second state.

2. (canceled)

3. (canceled)

4. (canceled)

5. The device of claim 1, wherein the first wireless transceiver is inactive while the device is in the first state, and wherein the first wireless transceiver is active while the device is in the second state.

6. The device of claim 1, wherein the first wireless transceiver transmits data only while the device is in the second state.

7. The device of claim 1, wherein the first state is a lower-power standby state, and wherein the second state is a higher-power operational state.

8. The device of claim 7, wherein, in the standby state, the device does not transmit data via the first wireless transceiver, and wherein in the operational state, the device transmits data via the first wireless transceiver.

9. The device of claim 1, wherein the device is configured to transition between the first state and the second state after a trigger event.

10. The device of claim 9, wherein the trigger event comprises at least one of a measurement of a physiological parameter that falls above or below a predetermined threshold, a measurement of a physiological parameter that falls outside of a predetermined range, elapsing of a predetermined time, or receiving a modulation signal from an external device.

11. (canceled)

12. The device of claim 9, wherein the device is configured to transition from the second state back to the first state after a second trigger event.

13. The device of claim 12, wherein the second trigger event comprises at least one of a measurement of a physiological parameter that falls within a predetermined range, a measurement of a physiological parameter that falls above or below a predetermined threshold, elapsing of a predetermined time, or completion of data transmission to an external device.

14. The device of claim 1, wherein, in the first state, at least one sensor has a first sampling frequency and, in the second state, the at least one sensor has a second sampling frequency greater than the first.

15. (canceled)

16. (canceled)

17. (canceled)

18. A method comprising:
receiving, at a first wireless transceiver of an implantable monitor, a first signal over a first communication link;
after receiving the first signal, transitioning the implantable monitor from a low-power first state to a higher-power second state;
sensing, at one or more sensors of the implantable monitor, at least one physiological parameter while implanted within the patient; and
while in the second state, transmitting data associated with the at least one physiological parameter to one or more external devices over a second communication link via a second wireless transceiver different from the first.

19. (canceled)

20. (canceled)

21. (canceled)

22. The method of claim 18, wherein the first wireless transceiver is inactive while the device is in the first state, and wherein the first wireless transceiver is active while the device is in the second state.

23. The method of claim 18, wherein the first wireless transceiver transmits data only while the device is in the second state.

24. The method of claim 18, wherein the first state is a lower-power standby state, and wherein the second state is a higher-power operational state.

25. The method of claim 24, wherein, in the standby state, the device does not transmit data via the first wireless transceiver, and wherein in the operational state, the device transmits data via the first wireless transceiver.

26. The method of claim 18, wherein the device transitions between the first state and the second state after a trigger event.

27. The method of claim 26, wherein the trigger event comprises at least one of a measurement of a physiological parameter that falls above or below a predetermined threshold, a measurement of a physiological parameter that indicates a rate of change that is above or below a predetermined threshold, a measurement of a physiological parameter that falls outside of a predetermined range, elapsing of a predetermined time, or receiving a modulation signal from an external device.

28. (canceled)

29. The method of claim 26, further comprising transitioning from the second state back to the first state after a second trigger event.

30. The method of claim 29, wherein the second trigger event comprises at least one of a measurement of a physiological parameter that falls within a predetermined range, a measurement of a physiological parameter that falls above or below a predetermined threshold, elapsing of a predetermined time, or completion of data transmission to an external device.

31. The method of claim 18, wherein, in the first state, at least one sensor has a first sampling frequency and, in the second state, the at least one sensor has a second sampling frequency greater than the first.

32. (canceled)

33. (canceled)

34. (canceled)
